(19) United States
(12) Patent Application Publication
(10) Pub. No.: US 2017/0333604 A1
(43) Pub. Date: Nov. 23, 2017

(54) READY-MADE BIOMEDICAL DEVICES FOR IN VIVO WELDING

(71) Applicants: YISSUM RESEARCH DEVELOPMENT COMPANY OF THE HEBREW UNIVERSITY OF JERUSALEM LTD, Jerusalem (IL); HADASIT MEDICAL RESEARCH SERVICES & DEVELOPMENT LIMITED, Jerusalem (IL)

(72) Inventors: Daniel COHN, Jerusalem (IL); Allan I. BLOOM, Neve Ilan (IL)

(21) Appl. No.: 15/525,476

(22) PCT Filed: Nov. 10, 2015
(86) PCT No.: PCT/IL2015/051089

§ 371 Date: May 9, 2017

Related U.S. Application Data
(60) Provisional application No. 62/077,494, filed on Nov. 10, 2014.

(51) Int. Cl. A61L 31/14; A61L 31/06; A61F 2/07; A61F 2/945; A61L 31/02; A61F 2/82

(52) U.S. Cl. CPC A61L 31/14; A61F 2/945; A61L 31/022; A61L 31/06; A61L 31/146; A61F 2/07; A61F 2002/826; A61F 2210/0014; A61F 2220/0058; A61F 2210/009

(57) ABSTRACT

Disclosed herein is a unique family of medical implants which are engineered outside of a subject's body into a form which may be manipulated in vivo. The implants comprise a region of at least one weldable material which allows welding of the implant to a polymeric material introduced into the body prior to, together with or after the implant has been positioned.

### TECHNOLOGICAL FIELD

The invention generally relates to devices engineered for in vivo welding.

### BACKGROUND ART

[1] WO 2011/007352

### BACKGROUND

Current methods for in vivo assembly of medical devices involve mainly mechanical assembly of metallic implant segments one to another. In vivo assembling methods involving association of polymeric segments mainly involve the use of a biocompatible adhesive or other means which meet the operating requirements of the physiological environment in which the implant is assembled, without risking being injurious to tissue and organs or causing discomfort to the patient.

In vivo welding of polymeric materials for the construction of implants has been disclosed in WO2011/007352 [1]. However, the technology disclosed does not permit welding to surfaces which are not “weldable”, such surfaces being for example metallic surfaces, ceramic surfaces and polymeric surfaces which are resistant to in vivo softening.

### GENERAL DESCRIPTION

The invention disclosed herein provides a novel family of implantable systems, medical and surgical instrumentation and accessories, which are engineered for in vivo welding. As known in the art, many of the implantable devices are based on metallic skeletons or backbones or scaffolds which assist the devices in maintaining a desired shape over time, thus preventing the device from collapsing, expanding, or bending. In vivo manipulation of such devices, e.g., having a metallic surface or regions, or devices having surface regions composed of materials (such as ceramic materials, certain polymeric materials and certain bioactive materials) that cannot be associated in vivo to other materials, for the purpose of improving their performance or adding a new aspect to their performance such as reinforcing or rendering them more robust or for attaching them to additional device segments of the same or different materials is typically not possible. Where in vivo manipulation is possible, the need for interaction with the surface material, e.g., metallic material or any other material that requires manipulation, as described herein, limits the possible material-to-material interactions, as such interactions cannot typically be achieved in vivo without risking being injurious to tissue and organs or causing discomfort to the patient.

The inventors of the invention disclosed herein have developed a unique family of medical implants which are engineered outside of the subject's body into a form which may be manipulated in vivo. The implants of the invention typically comprise a region of at least one weldable material which allows welding of the implant to a polymeric material introduced into the body prior to, together with or after the implant has been positioned.

The inventors have further developed a kit comprising two or more implant segments, each of said segments is suitable for assembly in vivo, to construct or assemble in the subject's body the complete implant. As explained herein, each of the implant segments comprised in the kit has at least one material region which is suitable for in vivo welding. The construction or forming of the implant segments is achieved ex vivo.

Thus, in a first aspect the invention provides an implantable article engineered to receive (be associated with, welded to or fused) onto at least a region thereof an element comprising a polymeric material, wherein the receiving (association or welding or fusing of said engineered implant device or system to said element) is achieved in vivo.

In a further aspect, the invention provides an implantable article constructed or comprising a material selected from a metal, a ceramic material and a polymer having a softening temperature above 40° C. or above 45° C., said article being modified to receive, by welding, onto at least a region thereof an element comprising a polymeric material, wherein welding is achieved in vivo. In some embodiments, the at least a region is associated with a polymeric material having been associated with said region ex vivo, wherein said element being capable of welding to said element in vivo. In some embodiments, said polymeric material is associated with a region on said implantable article which is resistant to in vivo welding (namely on the material which is selected from a metal, a ceramic material and a polymer having a softening temperature above 40° C. or above 45° C., as defined).

The invention further provides an implantable article constructed or comprising a material selected from a metal, a ceramic material and a polymer having a softening temperature above 40° C. or above 45° C., said article being rendered weldable ex vivo, for use in a process of in vivo welding of said article to at least one element comprising a polymeric material.

The invention further provides an implant when positioned in an animal body, the implant comprising a material region constructed or comprising a material selected from a metal, a ceramic material and a polymer having a softening temperature above 40° C. or above 45° C., said material region being associated with a first material, said first material being associated with a second material, wherein association of said region with the first material is achieved ex vivo and wherein association of said first material with said second material is achieved in vivo.

As may be understood, as used hereinabove, the “at least a region” comprises a material which is resistant to in vivo welding, namely a material which does not undergo material softening at physiologically acceptable temperatures to accept welding to another material. This material may be selected from metallic materials, ceramic materials and polymeric materials having softening temperatures above 40° C. or above 45° C. As noted herein, the resistance to welding is derived from the inability of a material, as defined, to exhibit the required mobility at physiological conditions, e.g., polymer chains, at a given temperature. The mobility and, therefore, the ability of the material, e.g., polymer chains to inter-diffuse with the material, e.g., polymer chains of the other material, e.g., another polymeric article, depend on the composition and molecular weight of the polymer. Where the material is a polymeric material, the former is determined by the rigidity of the backbone, the bulkiness and stiffness of the side groups, the strength and frequency of hydrogen bonds present between the chains. While temperature may increase material mobility, the temperatures suitable for application must be restricted to those physiologically acceptable.

In another aspect, the invention provides an implantable article rendered weldable ex vivo for use in a process of in vivo welding of said article to at least one element comprising a polymeric material.

In other words, the invention provides an implant which following its implantation in a subject's body comprises a material region associated with a first material, said first material being associated with a second material, wherein association of said region with the first material is achieved ex vivo and wherein association of said first material with said second material is achieved in vivo. As the implant is an article which may be implanted, as used herein, the material region “associated with a first material” is a material region of an article prior to ex vivo welding; the “first material” is a weldable polymeric material which association with the article region is achievable ex vivo, as defined; and the “second material” is a material of a further implant which in vivo welding to the implanted weldable article is desired.

In another aspect, the invention provides a kit for in vivo assembly of an implantable article, the kit comprising two or more article segments suitable for assembly into said article, in vivo, at least one of said article segments being comprised of a material selected from a metal, a ceramic material and a polymer having a softening temperature above 40° C. or above 45° C., each of said article segments having at least one material region selected of a weldable material, the kit further comprising instructions for assembling said article in vivo.

A further kit is provided for in vivo assembly of an implantable article, the kit comprising two or more article segments suitable for assembly into said article, in vivo, at least one of said article segments comprising a material selected from a metal, a ceramic material and a polymer having a softening temperature above 40° C. or above 45° C., said at least one of said article segments having at least one material region selected of a weldable material, the kit further comprising instructions for assembling said article in vivo.

Further provided is a kit or an article of manufacture or a commercial package for assembling in vivo an implantable article (e.g., an implant), the kit comprising a plurality (two or more) of article segments (parts, elements, building blocks) suitable for assembly in vivo, into said article, each of said article segments having at least one region selected of an in vivo weldable material, and instructions for assembling said article in vivo.

Each of the kits of the invention may further comprise one or more polymeric elements which are weldable in vivo to at least one other of the segments provided in the kit.

In each of the kits according to the invention, one or more of the segments of the kit may be constructed of a material selected from a metal, a ceramic material and a polymer having a softening temperature above 40° C. or above 45° C., and further have at least one material region selected of a weldable material. Alternatively, at least one of the segments may be constructed entirely of a polymeric material having a softening temperature below 40° C., thus being suitable for in vivo welding.

In some embodiments, each of the article segments may be constructed of a material which is weldable in vivo, as defined herein, or which is associated post-manufacture with an element comprising a polymeric material which may be welded in vivo to another of said article segments. The article segments are welded in vivo via each of said at least one region selected of a weldable material which is present on each segment. In some embodiments, each article segment comprises a single region of at least one weldable material. In some embodiments, each article segment comprises more than one region of at least one weldable material. In some embodiments, each article segment is appended or associated with an element of at least one weldable material, said element having been associated with said article segment ex vivo.

In some embodiments, one or more of the article segments is engineered and manipulated ex vivo to receive (be associated with, welded to or fused) thereonto at least one element comprising a polymeric material, and each of the remaining article segments comprises a single region of at least one weldable material.

In some embodiments, one or more of the article segments is engineered and manipulated ex vivo to receive (be associated with, welded to or fused) thereonto at least one element comprising a polymeric material, and each of the remaining article segments is of at least one weldable material.

Each of the article segments comprised in a kit of the invention is provided in a form, structure, size, shape and material constitution suitable for a desired purpose. Thus, each kit of the invention may be tailored for a different application or for achieving a different medical purpose.

As used herein the implantable article of the invention, is an implant, device or system, used herein interchangeably, including prostheses, instruments and accessories, and any other object, including optionally a bioactive material, which is suited for positioning or implanting in a subject's body—human or animal. The article may be suited to be permanent or temporary, and is selected to be engineered into a form suitable to undergo in vivo association, fusion or welding.

Numerous suitable implantable articles, addressing various needs may be engineered or manipulated to be rendered in vivo weldable. Such articles may be selected from devices traditionally utilized in areas such as orthopedics, in the cardiac arena, in vascular surgery, in the respiratory system, throughout the GI tract, along the urinary system, in general surgery, in ophthalmology, plastic surgery, neurosurgery, gynecology, surgical fields, such as wound closure.

The implantable article, by virtue of its known use, is constructed of a material which is typically inert to association with other materials, and therefore exhibits reduced or diminished capability to associate with another material in vivo, unless extreme conditions are employed or suitable mechanical manipulation is used to render at least a region of its surface weldable. While the in vivo association between the article of the invention and an element comprising a polymeric material is generally referred to as “in vivo welding”, it should be clear that the association is by no means limited to any one type of association. Typically, the association is not mechanical, but rather involves fusing the two components (namely a region on the implantable article and the element of at least one polymeric material which is delivered for in vivo welding) to form one continuous article.

Generally speaking, and without wishing to be bound by theory, the term “welding”, as used in context of the invention, is defined as a process, typically a temperature aided process, and/or a process aided by addition of suitable liquids, and/or a process aided by application of pressure, or any one of temperature and pressure, whereby the materials of two or more components, are caused to blend or fuse or intermingle inside the subject's body, resulting in a suitably strong connection between the two or more components. In vivo welding is performed at any acceptable temperature that does not harm the patient, locally or systemically, and which entails any part of each of the devices being welded/fused together.

Thus, to achieve welding or association or fusion between the two components, each component should have at least a region of a weldable material; welding will take place in vivo through a weldable region on each component. The implantable article is engineered or manufactured or manipulated by associating thereto, ex vivo, or forming thereon, ex vivo, at least one region of a weldable material, thereby rending the article in vivo weldable; namely, being suited for undergoing welding or fusion or association with at least one article after the implantable article has been positioned in the body.

Any region of the implantable article being non-in vivo weldable (i.e., being of a material which cannot be associated with, fused with or welded to another material) can be rendered in vivo weldable by the addition of an in vivo weldable component to it.

The weldable component is typically a polymeric material having a softening temperature below 45° C. or below 40° C. In some embodiments, the polymeric material has a softening temperature between body temperature (in vivo temperature), ca. 37° C. and 40° C.

The implantable article which cannot be welded in vivo, namely which is non-in vivo weldable, before said ex vivo manipulation, may be made of a polymeric material having a softening temperature above 40° C. or above 45° C., a metal, a ceramic material, carbons and/or a bioactive material. In some embodiments, the article to be rendered in vivo weldable is made of or comprises at least one metallic material. In other embodiments, the article to be rendered in vivo weldable is made of or comprises at least one polymeric material, being apriori non-weldable. In other embodiments, the article to be rendered in vivo weldable is made of or comprises at least one bioactive material.

Where the article is made of or comprises at least one polymeric material, the material may be a polymeric material having a high softening temperature, T_{g} or Tₘ, being at most 70-80° C.

In some embodiments, the polymeric material which is resistant to welding has a softening temperature above 40° C.

In some embodiments, such polymeric materials may be selected from polyethylene terephthalate PET, polybutylene terephthalate PBT, polytetrafluoroethylene PTFE, fluorinated ethylene propylene FEP, polyamides Nylons, polyethylene PE, polypropylene PP, styrene butadiene styrene, SBS; polymethylmethacrylate PMMA, polyethylmethacrylate PEMA, polyether amide PEBAX, polyurethanes PUs, polycarbonates PCs, polyethylene adipate PEA, polybutylene succinate PBS, polybutylene adipate PBA, polyglycolic acid PGA, poly (L)lactic acid P(L)LA, silicone-based polymers and others.

The region formed on the implantable article through which association, fusion or welding with said element may be added and/or incorporated and/or attached and/or welded and/or woven and/or knitted and/or braided and/or coated, ex vivo, to a surface region of the implantable article. The material from which the weldable region is formed may be selected based on the application method, or on the process by which its association to the implantable article is to be achieved or on any other property required, such as the strength or durability of the association. Generally speaking, the region may be formed in or on any region or part or feature of the implantable article, isotropically or anisotropically, and/or following any configuration, and may be of any size and geometry, and any such region may be positioned differently.

The region so formed may be of the same material as a polymeric material from which the element to be welded in vivo with the pre treated implanted device with is formed, or may be of a different material.

In some embodiments, the region is made of or comprises a polymeric material which may or may not comprise at least one additional material e.g., a monomer or oligomer, which undergoes polymerization and/or cross-linking In some embodiments, the region is made of or comprises a polymeric material which may or may not comprise at least one bioactive material.

In some embodiments, the polymeric material is a polymer formed of a monomer selected from vinyl monomers such as acrylate, methacrylate, diacrylate and a dimethacrylate. In some embodiments, the polymeric material comprises at least one monomeric material selected from vinyl monomers such as acrylate, methacrylate, diacrylate and a dimethacrylate.

In some embodiments, the polymeric material is a polymer formed of an oligomer selected from ethylene glycol dimethacrylate EGDMA, triethylene glycol dimethacrylate TEGDMA, polyethylene glycol PEG 600diacrylate, polypropylene glycol PPG500diacrylate, PEG 600dimethacrylate, PPG500dimethacrylate, double-bond end capped PEG/PPG diblocks and triblocks, low molecular weight polyamides, polyurethanes and polyesters. In some embodiments, the polymeric material comprises an oligomer selected from EGDMA, TEGDMA, PEG 600diacrylate, PPG500diacrylate, PEG 600dimethacrylate, PPG500dimethacrylate, double-bond end capped PEG/PPG diblocks and triblocks, low molecular weight polyamides, polyurethanes and polyesters.

In further embodiments, the material from which the weldable region is formed comprises a polymer selected from polymethyl methacrylate, poly(styrene-co-methyl methacrylate), polycaprolactone-polyurethane (e.g., CLUR2000) as a polymer and 2-hydroxyethyl methacrylate (HEMA) as a monomer. In further embodiments, the polymer is polycaprolactone (e.g., PCL80K) and ethylene glycol dimethacrylate (EGDMA) is the in vivo polymerizable monomer.

In further embodiments, the material from which the weldable region is formed comprises a polymer and a hydrophilic compound which plasticizes (reduces the stiffness of) the polymer. Optionally, the hydrophilic compound is a polyalkylene glycol (e.g., polyethylene glycol). The hydrophilic compound optionally has a low molecular weight, e.g., optionally less than 2000 Da, optionally less than 1000 Da, and optionally less than 500 Da.

In some embodiments, the material from which the weldable region is formed is a hydrophobic plasticizer, such as polypropylene glycol (PPG) and PEG.

In some embodiments, the material from which the weldable region is formed is a polymeric system comprising a first material, polymeric or not, having a first functional group and a further material, polymeric or not having a second functional group, wherein the first functional group and the second functional group are capable of reacting with one another upon stimulation, e.g., thermal stimulation or radiation, to form a new material, in some instances a polymeric material, and yet other instances, cross-links are formed, such that the obtained polymeric material is a cross-linked polymer. In such embodiments, the resulting article may display properties, such as enhanced toughness or tunable hydrophilicity. In the case when cross-linking is effected, a polymeric material with stiffness higher than the polymeric system is formed. Stimulation for effecting such a cross-linking include, but is not limited to, chemical stimulation and/or thermal stimulation (e.g., subjecting the polymeric system to the presence of a suitable catalyst; subjecting a polymeric system that already comprises a suitable catalyst to physiological conditions, e.g., 37±5° C. and/or aqueous environment; subjecting the polymeric system to physiological conditions, e.g., 37±5° C. and/or aqueous environment; or irradiative stimulation, e.g., light in the visible or UV spectral range.

The aforementioned polymeric systems or polymeric materials, recited in reference to materials from which the weldable region is made of, optionally comprise a polymer having both the first and second functional groups, such that the polymer is capable of cross-linking with itself. Such a system would be a mono-component system or a bi-component system, in case where a catalyst is required for promoting cross-linking Alternatively or additionally, the system comprises a first functional group on one polymer and a second functional group on a different polymer, such that the system comprises a pair of polymers capable of cross-linking with one another. Such a system would be a bi-component system or a tri-component system, in cases where a catalyst is required for promoting cross-linking

Examples of pairs of functional groups capable of reacting with one another include an azide and an alkyne, an unsaturated carbon-carbon bond (e.g., acrylate, methacrylate, maleimide) and a thiol, an unsaturated carbon-carbon bond and an amine, a carboxylic acid and an amine, a hydroxyl and an isocyanate, a carboxylic acid and an isocyanate, an amine and an isocyanate, a thiol and an isocyanate. Additional examples include an amine, a hydroxyl, a thiol or a carboxylic acid along with a nucleophilic leaving group (e.g., hydroxysuccinimide, a halogen).

In some embodiments, the first and second functional groups comprise an azide and an alkyne. The two functional groups may combine to form a triazole ring, by a mechanism referred to as “click” chemistry. Formation of a triazole ring constitutes cross-linking (e.g., between two polymers and/or within a single polymer), which increases a stiffness of the polymeric system. Optionally, a stimulation which results in such cross-linking comprises exposure to a catalyst of a click reaction. Copper compounds (e.g., Cu(I) compounds) are exemplary catalysts of a click reaction.

In some embodiments, the first and/or the second functional groups can be latent groups, which are exposed upon said stimulation, such that cross-linking is effected once a latent group is exposed. Exemplary groups include, but are not limited to, functional groups as described hereinabove, which are protected with a protecting group that is labile under the stimulation. Examples of labile protecting groups and the forms of stimulation to which they are susceptible include carboxylate esters, which may hydrolyzed to form an alcohol and a carboxylic acid or by exposure to an esterase and by exposure to acidic or basic conditions; silyl ethers such as trialkyl silyl ethers, which can be hydrolysed to an alcohol by acid or fluoride ion; p-methoxybenzyl ethers, which may be hydrolysed to an alcohol, for example, by oxidizing conditions or acidic conditions; t-butyloxycarbonyl and 9-fluorenylmethyloxycarbonyl, which may be hydrolysed to an amine by a exposure to basic conditions; sulfonamides, which may be hydrolysed to a sulfonate and amine by exposure to a suitable reagent such as samarium iodide or tributyltin hydride; acetals and ketals, which may be hydrolysed to form an aldehyde or ketone, respectively, along with an alcohol or diol, by exposure to acidic conditions; acytals (i.e., wherein a carbon atom is attached to two carboxylate groups), which may be hydrolysed to an aldehyde of ketone, for example, by exposure to a Lewis acid; orthoesters (i.e., wherein a carbon atom is attached to three alkoxy or aryloxy groups), which may be hydrolysed to a carboxylate ester (which may be further hydrolysed as described hereinabove) by exposure to mildly acidic conditions; 2-cyanoethyl phosphates, which may be converted to a phosphate by exposure to mildly basic conditions; methylphosphates, which may be hydrolysed to phosphates by exposure to strong nucleophiles; phosphates, which may be hydrolysed to alcohols, for example, by exposure to phosphatases; and aldehydes, which may be converted to carboxylic acids, for example, by exposure to an oxidizing agent.

In some embodiments, the polymeric system comprises a polymer and a compound which reacts with the polymer upon stimulation, to produce the polymeric material. The polymeric material may be, for example, a cross-linked form of the polymer, a derivative of the polymer (e.g., a chain extension derivative of the polymer) or a co-polymer (either non cross-linked or cross-linked). Optionally, the compound is a monomer or oligomer which undergoes polymerization upon stimulation. The compound undergoing polymerization may react with a polymer originally present in the polymeric system, for example, by cross-linking with the polymer as a result of polymerization of the monomer or oligomer (e.g., wherein a functional group in the original polymer attaches to a monomer or oligomer during polymerization) and/or by forming a copolymer with the polymer originally present in the system (e.g., by chain extension of the original polymer).

Suitable stimulations for effecting the herein-described interactions between a polymer and the monomer or oligomer include, but are not limited to, thermal stimulation (e.g., exposing to a physiological temperature to a supra-physiological one); chemical stimulation (e.g., for exposing a latent functional group, as described herein); and/or optical stimulation (e.g., for exposing a latent functional group and/or for initiating polymerization).

Examples of monomers suitable for use in the context of the embodiments include, but are not limited to, acrylates, methacrylates, diacrylates and dimethacrylates, as well as other monomers that polymerize or cross-link at mild conditions such as physiological conditions or biocompatible conditions.

Optionally, the compound is a cross-linker capable of cross-linking the polymer upon stimulation. Suitable cross-linkers include compounds with two or more reactive functional groups (e.g., thiol, amine, unsaturated bond, azide, alkyne and optionally any functional group described herein with respect to the abovementioned first and second functional groups) capable of reacting with a functional group of a polymer, for example, a dithiol, a diamine, an aminothiol, an amino acid (e.g., lysine, cysteine), an oligopeptide, a bis(azide), a dialkyne, a diacrylate and a dimethacrylate, and combinations thereof. The functional groups of the cross-linker may be the same (e.g., as in a dithiol and a diamine) or different (e.g., as in an aminothiol). The cross-linker may be a small molecule (e.g., a monomer) or a large molecule (e.g., an oligomer or a polymer).

In some embodiments, the compound which reacts with the polymer and/or the polymer itself comprises functional groups which are latent groups, which are exposed upon the stimulation, such that cross-linking is effected once a latent group or groups are exposed. Exemplary latent groups and suitable types of stimulation for exposing the latent groups are described hereinabove which are protected with a protecting group that is labile under the stimulation.

In an exemplary embodiment, the cross-linker is a bis(azide), such as an azide-terminated polymer, and the polymer being cross-linked comprises alkyne groups.

Cross-Linking May Result from a Click Reaction, as Described Herein.

The components of the polymeric systems recited herein as materials from which the weldable region(s) is made from render the polymeric component or system in vivo weldable and may further enhance the mechanical properties and/or impart to the weldable region any other advantage. The advantages of the engineered articles may be enhanced by manipulating the material crystalline form. In general, embodiments relating to such a manipulation involve stimulation that effects transformation from an amorphous form to a semi-crystalline form or crystalline form, or from a semi-crystalline form to another semi-crystalline form with a higher degree of crystallinity. Polymeric systems useful in these embodiments are therefore selected so as to undergo crystallization of a polymer upon stimulation. Alternatively, in several embodiments, a semi-crystalline polymer may be stimulated to become less crystalline or amorphous, generating a liquid component that will improve the in vivo weldability of the polymeric component. Crystallinity of a substance can be determined by methods well known in the art (e.g., by measuring X-Ray diffraction or Differential Scanning calorimetry).

In some embodiments, the polymeric system comprises a substance (e.g., a substance comprising a polymer) in an amorphous form. Upon stimulation, at least a portion of the amorphous form undergoes crystallization to form a crystalline or semi-crystalline form of the substance. In such embodiments, the crystalline or semi-crystalline form of the substance provides the polymeric system with more stiffness than does the amorphous form of the substance. Optionally, a polymer undergoes crystallization, such that the polymeric material is a crystalline or semi-crystalline polymer which is stiffer than the amorphous form of the polymer.

Optionally, the substance in an amorphous form comprises a compound (e.g., a polymer) characterized by a crystallization temperature at or slightly below 37° C. (e.g., in a range of 30-37° C.), such that exposure to physiological temperature provides stimulation for crystallization. Exemplary polymers exhibiting a suitable crystallization temperature include, but are not limited to, polycaprolactone-polyurethanes (e.g., CLUR polymers).

As may be known in the art, CLUR polymers are formed by reacting OH terminated PCL segments with a diisocyanate, such as hexamethylene diisocyanate (HDI), whereby a polyester urethane is formed. The molecular weights of the OH terminated PCL segments range between 1,000 and 20,000. Thus, in some embodiments, the molecular weight of a selected CLUR polymer is between 5,000 and 35,000. An amorphous state of such polymers may be obtained, for example, by melting the polymer and then rapidly quenching the polymer.

Additionally or alternatively, crystallization of a substance in amorphous form is enhanced by absorption of water into the substance, such that exposure to an aqueous environment in a body provides stimulation for crystallization. Without being bound by any particular theory, it is believed that absorbed water can induce crystallization of a polymer by increasing a mobility of the polymer chains (e.g., by acting as a plasticizer) so as to allow reordering of molecules to take place, thereby inducing crystallization, as described herein as WINC (water induced crystallization). Optionally, the substance comprises a cross-linked polymer in a non-crystalline (e.g., amorphous or semi-crystalline) form, the polymer comprising degradable cross-links which interfere with crystallization of the polymer. Stimulation comprises degrading (e.g., hydrolysis) of the cross-links, and at least a portion of the polymer undergoes crystallization following (partial or total) degradation of the cross-links

In some embodiments, the polymeric system comprises a substance (e.g., a substance comprising a polymer) in a semi-crystalline form. Upon stimulation, at least a portion of the semi-crystalline form undergoes crystallization to form a crystalline form of the substance, which is characterized by higher stiffness.

In some embodiments, the polymeric system comprises a substance in a non-crystalline form (e.g., an amorphous form or a semi-crystalline form) and an additional hydrophilic substance. Such a system, when exposed to an aqueous environment (e.g., a blood vessel) as stimulation, can undergo swelling due to the non-crystalline nature of the polymer and/or the hydrophilic nature of the additional substance. As noted hereinabove, such a swelling results in decreased stiffness. Upon expansion, and possibly an additional stimulation, as described hereinabove, the additional substance is released from the system, the latter “loses” its hydrophilic nature such that swelling is reduced, and is subjected to “Solvent Induced Crystallization” as described hereinabove.

In some embodiments, the material from which a weldable region is formed is selected amongst polymers such as polyamides such as polyhexamethylene adipamide, polyoctamethylene adipamide, polynonamethylene adipamide, polyhexamethylene sebacamide, polyoctamethylene sebacamide, polyhexamethylene azelamide, and polyhexamethylene dodecanediamine; polyolefins such as Low Density Polyethylene and ethylene-octene co-polymers Exact8201 and Exact8230; polyesters such as polydecamethylene terephthalate; Silicone polymers, such as poly(di-p-tolylsiloxane); polyurethanes such as Biomer, Pellethane, Cardiothane, Biospan, Estane, Tecoflex, as well as polystyrene, polymethyl methacrylate, polyethyl methacrylate, polyvinyl chloride, polybutyl methacrylate and polypropyl methacrylate.

In some embodiments, the material is a polymer selected from hydroxy ethylmethacrylate, ethylene glycol dimethacrylate, diethylene glycol dimethacrylate, triethylene glycol dimethacrylate, tetraethylene glycol dimethacrylate, propylene glycol dimethacrylate, dipropylene glycol dimethacrylate, tripropylene glycol dimethacrylate, tetrapropylene glycol dimethacrylate, trimethylene dimethacrylate, propyl dimethacrylate, ethoxylated methacrylates, with the pendant PEG chain having different molecular weights, t-butyl methacrylate, PEG diacrylates of various molecular weights, PEG dimethacrylates of various molecular weights, PEG dithiols of various molecular weights, PEG di-hydroxy succinimide of various molecular weights, PEG maleimide of various molecular weights, PPG diacrylates of various molecular weights, PPG dimethacrylates of various molecular weights, PPG dithiols of various molecular weights, PPG di-hydroxy succinimide of various molecular weights, PPG maleimide of various molecular weights, diisocyanates of various types and molecular weights, such as hexamethylene diisocyanate (HDI), PEGdiHDI of various molecular weights, PPGdiHDI of various molecular weights, and compounds that can participate in click reaction (e.g., azide-containing compounds or alkyne-containing compounds.

The polymer to be welded is a thermoplastic polymer characterized by a transition temperature (e.g., a glass transition temperature, a melting point) in a range of from about 45° C. to about 65° C. In some embodiments, the thermoplastic polymer encompasses a polymer, a co-polymer and/or a mixture of one or more polymers and/or copolymers and/or a semi-IPN or an IPN.

Examples of thermoplastic polymers according to some embodiments of the invention include, for example, a polyester, a polycarbonate, a polyurethane, a polyether urethane, a polyether carbonate, a polyester carbonate, a polyester urethane, a polyanhydride, a polyamide, a polyether amide, a polyether amide urethane, a polyolefin, a polyacrylate, a polymethacrylate, a halogenated polymer and a silicone polymer, and combinations and copolymers thereof, among numerous others.

In some embodiments, the thermoplastic polymer is a polycaprolactone (as an exemplary polyester), or a copolymer thereof, such as a polyester carbonate, a polyester urethane, a polycaprolactone-polyether copolymer (e.g., polycaprolactone-polyethylene glycol, polycaprolactone-polypropylene glycol, polycaprolactone-polytetramethylene glycol), and/or a copolymer of polycaprolactone and another polyester (e.g., polycaprolactone-polylactic acid).

Further examples of suitable polyesters include polybutylene succinate, polyethylene adipate, polyhexamethylene adipate, polyethylene sebacate, polybutylene sebacate and polyhexamethylene sebacate.

Examples of suitable polyamides include polybutylene sebacamide and polyhexamethylene sebacamide. Examples of suitable polyether urethanes include those consisting of polyethers such as, among others, polyethylene glycol, polytetramethylene glycol and polypropylene glycol of various molecular weights, and various diisocyanates, such as, without limitation, hexamethylene diisocyanate and lysine diisocyanate. The polymers may be synthesized following a one pot procedure or a two stages synthesis. The former case can be illustrated, among numerous others, by the polymerization of hexamethylene diisocyanate and polytetramethylene glycol 650. In the latter case, initially a macrodiisocyanate is formed by reacting a diol of different types and molecular weights, with a diisocyanate and then chain extending the macrodiisocyanate with any molecule able to react with the isocyanate moieties, such as, without limitation, with another diol, a diamine or a dicarboxylic acid.

Further examples of suitable polymethacrylate are, among many others, polybutyl methacrylate and its copolymers and blends.

The weldable region, according to some embodiments of the invention, may be conveniently expanded (e.g., increasing a diameter as required, in some instances by 200% or even more) by softening, optionally by heating it to a temperature typically in the 45-65° C. range. As further exemplified herein, the heating may be applied by inserting a balloon filled with a warm liquid (e.g., water, saline) into the tubular device. The balloon used to heat the thermoplastic polymer may optionally be used to expand the tubular structure, as further detailed herein below.

In some embodiments, the weldable region is of a polymer which is biodegradable. Examples of biodegradable polymers include aliphatic polyesters such as, without limitation, polycaprolactone and poly (DL) lactic acid, and copolymers of glycolic acid, lactic acid and caprolactone. Additional examples include, but are not limited to, aliphatic polyesters made of glycolide (glycolic acid), (DL) lactide (lactic acid), p-dioxanone, trimethylene carbonate, hydroxybutyrate, hydroxyvalerate, and also biodegradable co-polyamides, polydihydropyrans, polyphosphazenes, poly(ortho-esters), polycarbonates, poly(cyano acrylates), polyanhydrides and any combination thereof.

In some embodiments, the thermoplastic polymer is a non-biodegradable polymer.

Exemplary non-biodegradable thermoplastic polymers include, but are not limited to, silicone polymers, such as poly(di-p-tolylsiloxane (Tg=50° C.), poly(phenyl-p-tolyl siloxane) (Tg=40° C.), poly(di-phenylsiloxane) (Tg=40° C.); ethylene based polymers, such as different ethylene-octene co-polymers (also referred to as Exact; e.g., Exact9061 (m.p.=41° C.), Exact9071 (m.p.=50° C.), Exact9361 (m.p.=41° C.), and Exact9371 (m.p.=55° C.)); ethylene vinyl acetate copolymers; and polyesters such as, for example, polybutylene terthphtalate, polyhexamethylene terthphtalate, polyocta methylene terthphtalate, polyethylene adipate, polybutylene adipate, polyethylene pimelate, polybutylene pimelate, polypropylene adipate, polybutylene azealate, polyproylene azealate, and polyproylene sebacate; and polymethacrylates such as, without limitation, polypropyl methacrylate, polybutyl methacrylate, polypentyl methacrylate and combinations and copolymers thereof.

In some embodiments, the thermoplastic polymer is characterized in that it undergoes a decrease of its stiffness of at least 20% at a temperature ranging from 40° C. to 65° C.

The engineering or manufacturing or manipulation of a pre-made implantable article to render it weldable; namely capable of being in vivo welded occurs at any time prior to the implanting of the article in the body. The process involves associating, by any means known in the pertinent field, a region of the implantable article with an element which is selected of a material which exhibits viscoelastic behavior under physiological conditions, being typically polymeric materials or comprising such polymeric materials. Such materials have thermal transitions at temperatures that render them in vivo weldable and therefore allow welding them in any region, tissue or cavity in a subject's body, at physiologically acceptable temperatures (namely at any temperature that does not harm the patient, locally or systemically, as required by each specific indication, when applied as required to implement successfully the devices disclosed by this invention).

In some embodiments, the implantable article is associated with a “low softening temperature polymer”, having a softening temperature not exceeding 65° C. (degrees Celsius).

The at least one region which comprises or is of a weldable material, e.g., of at least one low softening temperature polymer may be a region of any size on the article surface. The region may be the complete surface of the article, any specifically selected region, or any plurality of such regions. The location of the region on the surface of the article, the number of such regions and their distribution and density on the article surface, depends, inter alia, on the article to be implanted, the type of association expected between the eventually implanted article and the later delivered element to be welded therewith, the site of welding, the selection of polymeric material used and other parameters.

In view of the shortcomings of the current clinical methodologies used in the treatment of a diversity of pathologies, such as, without limitation, the narrowing of the lumen of various organs and the formation of aneurysms, the implantation of a diversity of devices such as stents, stent/grafts and heart valves, among numerous others, the present inventors have devised and successfully practiced a novel methodology, aimed at overcoming several of the most important problems marring the performance and limiting the use of devices currently in clinical use, by in vivo welding the various components forming the device.

This invention discloses a novel type of implantable systems, including, among others, biomedical devices, implants and prostheses of any type, as well as medical and surgical instrumentation and accessories of any kind, displaying the unique ability of being in vivo weldable. When the in vivo welding is performed at the site of performance of the final device, they will be named in situ weldable.

The invention further provides a method for assembling an implantable article in vivo, the method comprising:
delivering into a body region a first segment of said article and positioning said segment at a desired position;
delivering, in sequence, one or more segments;
in vivo welding each segment to a previous segment by welding;

wherein at least of said article segments being constructed of or comprising a material selected from a metal, a ceramic material and a polymer having a softening temperature above 40° C., and having at least one material region selected of a weldable material, and

wherein welding is achieved by softening said at least one material region on each of two of the segments to be welded, permitting association of each of the article segments and article assembly.

In some embodiments, the first segment of said article is constructed of a material selected from a metal, a ceramic material and a polymer having a softening temperature above 40° C., and having at least one material region selected of a weldable material.

Among other numerous uses, the invention disclosed herein allows the construction of forming of devices, inside and/or on the body and/or in any of the cavities present on the surface of the body, often at their site of performance, from pre-formed components. Since the size of the final device is significantly larger than that of its components, this strategy allows implanting devices following especially minimally invasive procedures, through smaller orifices, thereby overcoming the constraints imposed by extremely hostile anatomies and minimizing trauma and pain, and speeding up patients recovery. Also, on many occasions, devices implanted in a body lack the stability and/or sealability required to ensure safe and efficacious performance. Clearly, having a hermetic seal between the different components of a multi-component device, and preventing leaks, is an extremely advantageous feature. Several devices often tend to migrate from their site of performance, this being an additional highly detrimental event, often with dangerous consequences. The devices taught by this invention successfully solve these and other important clinical problems, since they can be welded to other devices or instruments or accessories, and in some instances also to tissues. In some embodiments, the in vivo welding is aimed at moving, repositioning or retrieving the device.

Should insertion be required, means of insertion will include but will not be limited to percutaneous catheter directed image-guided placement as well as open surgery as well as any other surgical procedure that will allow the successful performance of these devices.

The devices disclosed hereby may be heated so they become in vivo weldable using means present inside the body, on or outside the body or in any cavity present at the surface of the body. In one embodiment of the invention, the component may comprise a material that will properly heat the component and/or parts of it and/or another component, and/or enhance the efficacy of the heating of the device. In one embodiment of this invention, among the means used inside the body to heat the component, a balloon filled with a suitably warm liquid or gas may be used. In another embodiment, an exothermic chemical reaction or a physical process may function, alone or in combination with other means, as the heating source required to perform the in vivo welding. In an exemplary procedure, the component will comprise a magnetic component, of any size, geometry and any other characteristic, embedded, partially or totally within the component, being heated from outside the body by an alternate magnetic field. In another exemplary procedure, when the devices disclosed hereby are heated by means outside the body, the in vivo weldable component/s will be heated using an ultrasound system.

As stated above, in some embodiments of this invention, the in vivo weldable component/s are made in vivo weldable by bringing it/them in contact with a liquid that suitably affects the polymer/s, so that it renders it/them in vivo weldable. Typically, this happens because the liquid lowers the relevant softening temperature of the previously non-in vivo weldable polymer to a physiologically acceptable temperature range, rendering it, therefore, in vivo weldable. In additional embodiments of the invention being disclosed hereby, the liquid required to render the device or any of its parts and/or any of its components to render it in vivo weldable, may by applied in the outside, prior to introducing the device into the body, while introducing it into the body, once introduced into the body or when at its site of performance, and combinations thereof. The liquid may be already present at the site before the deployment of the component/s, being natural or not, and/or may be added during or after the device or parts of it are deployed at the site.

In several embodiments of the present invention, the reactive liquid is present within the in vivo weldable component which, in some embodiments is inherently non-in vivo weldable but it is rendered in vivo weldable by the presence of the liquid, in any of its forms, as described hereby.

In yet other embodiments of this invention, the reactive liquid is able to react with selected reactive moieties, for example, without limitation, those present in the other/s in vivo weldable component/s of the device or other device or tissue.

Additionally, the liquid may be the result of a chemical reaction, for example, without limitation, one that generates water molecules, or small molecules resulting from a degradation process such as, without limitation, oligomers of various molecular weights. The liquid may also be the result of a physical phenomenon such as, without limitation, the loss of crystallinity of a semi-crystalline polymer. This can be illustrated by the following embodiment of this invention, where the initially non-in vivo weldable semi-crystalline polymer, or part of it, will become amorphous. While still being semi-crystalline, its supra-physiological Tₘ prevented it from being in vivo weldable, but now that it has become less crystalline or amorphous, it's infra- physiological T_{g} will allow the polymer to become in vivo weldable.

The polymeric components of this invention can be bio-durable, or partially or totally biodegradable. In the case of being totally or partially biodegradable, the biodegradable component/s may be localized at specific sections of the device or may be present throughout the whole device, and they may follow the same or different degradation mechanisms and/or kinetics.

According to some embodiments the devices do not contain any bioactive molecules, biological/cellular materials or drugs in the polymeric material. In other words, the implantable device rendered weldable does not contain, hold or is associated with any biological, material, drugs or bioactive materials, and the purpose of the polymeric material is merely for in vivo welding and not for drug/ bioactive material release or delivery.

In some instances, the devices disclosed hereby may also comprise or contain bioactive molecules or any other type of material displaying biological activity, including cells, that will then, in due time, be released in a programmed manner Among numerous others, the material displaying biological activity will be, among many others, without limitation, drugs of any type, peptides, proteins, enzymes, growth factors, saccharides and polysaccharides, glycosoaminoglycans, lipoproteins, DNA and DNA-related material, any material containing genetic information, cells, hormones, vitamins, and combinations thereof. It is also an embodiment of the present invention that molecules of various types and/or having different purposes will be attached, chemically or physically or in any other form, and/or having different objectives, to the surface of the component. In yet another embodiment, molecules covering a broad range of molecular weights, biologically active or not, may be covalently bound to the surface, following diverse surface grafting schemes.

In some instances, the in vivo weldable devices disclosed hereby may also include cellular material.

It is also an embodiment of the present invention that the devices disclosed hereby may comprise more than one type of material, such as, among many other combinations, they may comprise a metallic component/s and a polymeric component/s, or a ceramic component/s and a polymeric component/s.

In vivo weldability can take place totally inside the body, as well as on the surface of the body, partially inside and partially outside the body, or in any of the several body surface cavities and combinations thereof.

Even though the in vivo welding may be performed at deployment, in some embodiments of the present invention, part or the whole of the in vivo welding of any two or more components may take place gradually over time, and/or “on command”, in due time. If gradually, in one embodiment, among several others, the component may change one or more of its properties (chemical, physical, biological or any other and combinations thereof), so that the welding takes place gradually, or the changes take place gradually, until a given point is reached, that triggers the in vivo welding. If the in vivo welding will take place “on command”, the trigger for it may already be engineered into the system, or applied in vivo, within the body or ex vivo, in, on and/or the outside the body and/or in any of the several body surface cavities. In some embodiments of this invention, the heating trigger may be applied from the outside, for example, without limitation, applying an ultrasound heating field. In another embodiment of this invention, any of the components may comprise magnetic species, such as, without limitation, magnetic nanoparticles, and the application of an alternate magnetic field will result in their heating up, which in turn, will heat the component and render it in vivo weldable. This invention can be illustrated, without limitation and without detracting from the scope and generality of this invention in any form or shape, by endoluminal devices deployed in any tissue or organ having a lumen or a cavity, such as, among others, tubular tissues or organs such as the vasculature, the trachea, the bronchi, conduits in the nasal arena, the esophagus, the biliary duct, the intestines and the urethra, among numerous others. As stated above, the devices of the present invention can also be advantageously used exoluminally, between tissues and organs and at any other body site, as required.

In some instances the welding of the components may require pressure being applied to the components to be in vivo welded. Among others, the pressure may be applied by a balloon filled with an appropriate liquid and/or a gas at the temperature required, and the pressure and temperature may change as a function of time and position on and/or within the body. Additional sources of heat can be provided by other means such as, without limitation, an exothermic reaction, ultrasound and/or magnetic fields. Additional sources of pressure can be provided by other means such as, without limitation, by the use of shape memory polymers that will be programmed so that they will change their dimensions so that they will apply forces as required, when a specific trigger/s is/are applied. The trigger/s may be applied from the outside, for example, without limitation, applying an ultrasound heating field, or in the inside, for example, without limitation, the device absorbing liquid, typically water from the aqueous biological environment or any other liquid, or combinations thereof. In another embodiment, any of the components may comprise a magnetic species, such as, without limitation, magnetic nanoparticles or nanofibers, and the application of an alternate magnetic field will result in their heating up. Another embodiment of the present invention applies pressure by a stent, balloon expandable or self-expandable, that will apply the pressure required. Additionally, in some embodiments, said stent will be rendered in vivo weldable following various techniques, such as coating the struts or covering the whole stent with an in vivo weldable polymer and combinations thereof.

An additional type of trigger contemplated by the present invention pertains to a material/s that prevent/s a given chemical and/or physical phenomenon from taking place, and once this material undergoes a chemical or biochemical reaction and/or a physical process and/or biological process, another phenomenon that renders the component in vivo weldable, takes place. This embodiment of the invention is exemplified hereby by a hydrophobic biodegradable component, for example, a coating, which prevents or controls or minimizes water absorption by a component of the system. Once said hydrophobic biodegradable component, for example a coating, degrades and the coated component becomes exposed to aqueous medium, it starts absorbing water, changing its properties, such as, without limitation, its size and/or its mechanical properties, or allowing the contact of a specific species added to it with water, in due time, as a result of which, the component becomes in vivo weldable and, in some embodiments rendered with additional advantageous features. In some embodiments the phenomena take place spontaneously, due to the very presence of the device in vivo, while in other embodiments a specific trigger has to be applied, and combinations thereof.

One or more of the components, in vivo weldable or not, of the devices disclosed hereby may be permanent and biodurable, as dictated by the specifics of each clinical indication. Any of the components, in vivo or non-in vivo weldable of the devices disclosed hereby that is/are fully or partially temporary, will be so following different strategies and combinations thereof, such as, without limitation: [i] By using biodegradable materials, that will degrade over time, as required, following various mechanisms and kinetics, and combinations thereof; and/or [ii] By applying, in due time, an internal and/or external stimulus, such as, without limitation, temperature, pH, ionic strength, any chemically and/or biochemically active molecule, biological species, as well as electrical or magnetic fields, ultrasound, and combinations thereof, any of the stimuli may be applied once or several times and, if more than one stimulus is applied, they can be applied simultaneously or sequentially, and/or [Hi] By generating at least one of the component/s of the device so it consists of at least one slowly soluble material that, over time, will dissolve, as required. Additionally embodiments of this invention include the combined use of [i], [ii] and [hi], above.

The welded connection can be made transient, by using one, two or the three basic strategies described above, simultaneously or sequentially. The above relates also to any in vivo weldable material that was added to an initially non-in vivo weldable material, so it becomes in vivo weldable, such as, without limitation, coating the struts of a metallic stent with an in vivo weldable polymer. The above mechanisms can also be applied not only to the welded bond formed between two or more in vivo weldable components, regardless if they were inherently in vivo weldable or were rendered such by adding an in vivo weldable component, but also to the bond existing between any in vivo weldable component/s and the in vivo-non weldable component being rendered in vivo weldable by the in vivo weldable component added to it.

Any of the in vivo welded materials present in the final system, including those formed outside and/or on the body and/or in any cavity present at the surface of the body, prior to implantation, as well as those generated in vivo, at any stage of the procedure, can consist of one or more materials, of any type, size and shape, being present throughout the whole system or only at specific regions of it, distributed isotropically or anisotropically, on the surface and/or the bulk, for any purpose, including in vivo welding but also for any other objective, such as, without limitation, any chemical, physical, mechanical or biological purpose, and combinations thereof. In yet another embodiment of the invention disclosed hereby, the in vivo weldable device may consist of different materials that have different welding temperatures, so that the in vivo welding can be staged in time and/or spatially. In this embodiment, the materials having different welding temperatures may be distributed homogeneously throughout the component or be present at given regions of the component, such as, without limitation, on one or both sides of the component, at one or both ends, on one surface or both, among other configurations possible. It is another embodiment of this invention that different components and/or regions of the in vivo weldable component may become weldable following the application of different stimuli, such as, without limitation, temperature and the application of a suitable liquid, among any other combination of stimuli, if applied in vivo and/or from the outside of the body. The in vivo weldable component may also consist additional materials, for any other purpose besides being in vivo weldable, such as, without limitation, rendering the device with any chemical, physical, mechanical, optical, biological or any other type of properties, and combinations thereof.

In some embodiments, the in vivo weldable component is used throughout the whole procedure when it is already in vivo weldable, no specific stimulus needed to render it in vivo weldable.

In other embodiments, part of the balloon or the whole balloon may be in vivo weldable and may perform not only as an inflatable balloon but also as part or the whole in vivo weldable component. In this embodiment, the balloon is detached from its delivery system in due time and remains in its site of performance

While being able to be used at any site on or inside the body, as well as in any cavity present at the surface of the body, the devices of this invention can be used, without limitation and without detracting from the scope and generality of this invention in any form or shape, in endoluminal devices deployed along any tissue or organ having a lumen, such as, without limitation, the vasculature, the heart, the trachea, the bronchi, in the nasal arena, the mouth and the throat, in neural conduits, the esophagus, the stomach, the biliary duct, the intestines and the urethra, among many others.

In a specific embodiment, this invention can be illustrated, without limitation and without detracting from its scope and generality in any form or manner, by endoluminal devices used in the treatment of different pathologies throughout the cardiovascular system. In the heart, this can be illustrated, without limitation and without detracting from the scope and generality of this invention in any form or manner, by the treatment of diverse pathologies of the heart, such as, without limitation, or when implanting artificial heart valves, especially those that are implanted percutaneously. It is one embodiment of the present invention, to deploy components of the artificial heart valve sequentially, and weld them together in vivo. In one embodiment of this invention, the component comprising the metallic stent is deployed first, followed by the polymeric fabric, and then both are welded together in vivo, and in some instances in situ. The sequential deployment of the different components of the artificial heart valve and their in vivo welding together, allows reducing substantially the size of each of the components being implanted, significantly improving the outcome of the procedure and largely expanding its clinical applicability. In several embodiments of this invention, the order of deployment of the different components can vary, with any of them being first or second, or in any other order.

Along the vasculature, this can be illustrated, without limitation and without detracting from the scope and generality of this invention in any form or manner, by treatments of diverse diseases of blood vessels, including, without limitation, when aiming at blocking blood flow to a given site, for example, without limitation, when treating malignant tumors. In one of the embodiments of the invention, a metallic stent is first firmly deployed at the site of blockage, followed by the deployment of an in vivo weldable blocking component that will strongly weld to the stent, said blocking device combining in vivo weldability, with expandability and/or unfoldability and having a configuration or geometry, such as a closed cross-section part, typically distal, that will prevent blood from flowing downstream.

Also along the vasculature, this can be illustrated, without limitation and without detracting from the scope and generality of this invention in any form or manner, by treatments of diverse diseases of blood vessels, including, without limitation, when treating, for example, stenotic vessels or aneurysmal sacs. Among the latter, aneurysms of different types can develop and require treatment, and they include, without limitation, brain aneurysms, as well as thoracic, abdominal and peripheral aneurysms of various types and at different locations.

An aneurysm is a localized dilation of a blood vessel caused by the weakening and thinning of its wall, which represents a life-threatening pathology due to its potential for rupture. Since abdominal aortic aneurysms (AAA) are the deadliest of them all, the invention will be illustrated below for this dangerous illness.

The degenerative process whereby aneurysms are formed entails a profound histological change, whereby the vessel is stiffened and weakened substantially. While the elastin and collagen content of the healthy aorta are about 36% and 23%, respectively, aneurysmal tissues display much lower elastin content (around 6%), while the collagen content climbs up to 45%. As a result of these profound compositional changes, a marked change in mechanical properties ensues, with the longitudinal tensile strength decreasing from 160 kPa to 120 kPa and the stiffness increasing markedly, from 275 kPa up to 450 kPa.

Typically, the rupture of an abdominal aortic aneurysm (AAA) leads to almost immediate death, and in more than 80% of cases, its rupture is fatal. The mortality rate due to AAAs is so high because the process whereby aneurysms are created and expand is typically asymptomatic until burst occurs.

While there are additional contributing factors, such as high blood pressure, arteriosclerosis and smoking, contemporary theories collectively indicate that an underlying genetic factor is most probably involved. AAA appears in 5%-7% in the population over 60 years old, with a male:female ratio of 4:1. Approximately 45,000 AAA related operations are conducted each year in the USA. AAA rupture is the 13^{th} cause of death in the USA, with around 15,000 deaths per year. Among white men over 55 years, AAA ruptures ranks among the top 10 causes of mortality. The fact that the current AAA worldwide market is approximately 1 Billion dollars annually, illustrates the importance and prevalence of this clinical problem.

Until 1991, the only treatment available entailed a fully open surgical procedure, whereby the dilated segment of the artery was replaced by a Dacron or expanded PTFE arterial prostheses. An endoluminal device consisting of a vascular graft mounted on a metallic stent (‘stent graft’) and deployed intra-luminally at the aneurysmal site using a balloon, was implanted in 1991 for the first time. Once the stent graft is locked in place and the prosthesis is firmly positioned, the balloon is deflated and retrieved. This new minimally invasive technique, called EndoVascular Aneurysm Repair (EVAR), represented a breakthrough in the field both conceptually as well as technologically. Being a minimally invasive procedure, EVAR has several obvious advantages, the most important of which stems from the much shorter hospitalization and recovery periods required by this technique. In clear contrast to the open procedure, patients undergoing EVAR are usually discharged after two-three days in the hospital and have fully recovered after approximately two weeks. Clearly, therefore, whenever applicable, EVAR is the procedure of choice.

Unfortunately, though, this is not a technique of universal applicability and there are various factors that restrict substantially its use so that only about 60% of AAA repair treatments are done using the stent graft device.

The key limitations of stent grafts presently in clinical use stem from patients with complex anatomical constraints, dictated, for example, by the presence of narrow and/or convoluted and/or calcified access vessels, primarily the iliac arteries. Similarly, the lack of infra-renal landing zones and the fact that the aneurysm often involves not only the abdominal aorta but also compromises the iliac arteries, represent additional challenges for the existing EVAR devices. Also, typically, more than one endograft has to be deployed, with the different components being inter-connected by expanding the proximal end of one stent within the distal end of a second stent. These connections, relying primarily on radial force and “oversizing”, are often unstable, allowing blood leakage and relative motion of both components, which frequently lead to migration of the device and treatment failure.

Stent grafts of the prior art comprise a metallic stent to which a fabric has been bound to, typically by sewing the fabric to the struts of the metallic stent. As a result of the addition of the fabric to the stent, the profile of the bi-component device is rather large. In one embodiment of the invention disclosed hereby, a device has been developed so that the metallic stent and the polymeric component are implanted separately, via a sequential procedure, and welded together in vivo. This is an extremely advantageous feature of the endoluminal devices disclosed hereby, since it results in much smaller profiles, since the size of each of the components is markedly smaller than that of the final stent graft. The welding of the components of the stent graft can be done at any endoluminal site, from the insertion port, to the site of performance.

In some embodiments, the stent, rendered in vivo weldable by any of the techniques disclosed in this patent, such as, without limitation, by coating the struts of the stent with an in vivo weldable polymer, is deployed first, followed by the deployment of the in vivo weldable polymeric component, and welded together.

In another embodiment, the polymeric component is deployed first, followed by the metallic stent, the stent being rendered in vivo weldable by any of the techniques disclosed in this patent, such as, without limitation, by coating its struts with an in vivo weldable polymer, and both components are welded together in vivo.

In yet another embodiment, the polymeric component is deployed first, followed by the metallic stent, the stent being rendered in vivo weldable by any of the techniques disclosed in this patent, such as, without limitation, by coating its struts with an in vivo weldable polymer, followed by the deployment of a second polymeric component, that is welded in vivo to both the polymeric component already deployed and to the deployed stent, generating a sandwich with an external and an internal polymeric components, with the metallic stent in between, welded to the two.

In yet another embodiment, the polymeric component is deployed first, followed by a bare metal stent, followed by the deployment of a second polymeric component that is welded in vivo to the polymeric component already, generating a sandwich with an external and internal polymeric components welded between them, with the metallic stent in between them.

Each of the polymeric components may differ in their composition, dimensions, structure and configuration and/or have different chemical, physical, mechanical and/or biological properties, and combinations thereof, and they may also differ from the in vivo weldable polymer used to render the metallic component in vivo weldable. Each of the polymeric components may also contain biologically active species.

In some embodiments of this invention, any of the in vivo weldable components may be generated following a layer-by-layer approach, whereby extremely thin layers of the in vivo weldable component are deployed sequentially, and welded together. In some aspects of these embodiments, each of the layers may differ from the others in any of its chemical, physical, mechanical and biological properties, its size, especially its thickness, its orientation. Furthermore, some of them may contain specific additional components such as, without limitation, bioactive molecules, or cellular material, or magnetic material, among several others. This layer-by-layer approach constitutes yet another very advantageous feature of the invention disclosed hereby, since allow decreasing substantially the profile of the polymeric component, improving, therefore its performance and expanding its clinical applicability.

In some embodiments of this invention, the welding of any two in vivo weldable components may be performed following a layer-by-layer approach, or the components maybe welded together in parallel, in head-to-tail or in branched configurations, or any other spatial configuration, and combinations thereof, as dictated by the indication and the anatomy relevant. Among other advantageous features, this strategy enables to lower the relatively large profile of the existing unexpanded stent grafts, in some instances to as little as half their current unexpanded dimension.

In one embodiment, the in vivo weldable polymers, singularly or as plurality, are delivered using any suitable technique, such as, without limitation, aerosols of any kind, to a surface to be welded ex vivo or in vivo or in situ such that given an appropriate stimulus, such as, without limitation, on heating, the settled particulate material may coalesce via welding to a homogenous layer. In the case of a liquid aerosol, the continuous phase will be a suitable solvent that matches and exceeds the physiological and the environmental requirements for its appropriate use. The dispersed phase particles preferably have a diameter in the 1-1000 nanometer range and less preferably a diameter in the 1-1000 micrometer range. The layer may be of uniform or non-uniform in any of its characteristics, such as, without limitation, their chemical, physical, mechanical and biological properties, and combinations thereof, their thickness, isotropy or anisotropy, which may at a later stage be further welded to another surface, via any of the pathways disclosed hereby.

The completely new concept onto which the invention disclosed hereby is based allows generating medical devices having various advantageous features, such as overcoming the three largest and most challenging hurdles currently limiting the applicability of stent graft devices currently in clinical use:

(1) When the stent graft cannot be inserted due to narrow, tortuous or calcified access arteries. The largely reduced device profile and flexibility will overcome this limitation.

(2) Stabilization and sealing of overlapping modular components used when deploying stent grafts at branch points such as the aorto-iliac bifurcation or branch arteries arising from the aorta, such as the renal arteries. By welding them together via their polymeric component, as required, the stents will be firmly connected, spatially stabilizing the system and preventing migration phenomena, and blood leakage at the inter-stent connection will be precluded, and (3) Provision of a safe, efficacious and “off the shelf” solution for anatomies that lack infra-renal landing zones.

This innovative approach is applicable not only to these three key areas, which comprise most of the market that represent an important unmet clinical need, but for all endovascular procedures treating various pathologies, including aneurysm disease along the aorta and peripheral vessels. Furthermore, this technology will also impact other areas outside the vasculature, such as, without limitation, the heart, the urinary and respiratory systems and the GI tract.

In view of the shortcomings of the current clinical methodologies used in the treatment of aneurysms, particularly AAA, in one embodiment of the present invention the present inventors have devised and successfully practiced a novel methodology, which is aimed at largely improving their performance and widely expand their clinical applicability by in vivo connecting the constituents of any implanted device such as, without limitation, any endoluminal device, such as without limitation, endoluminal devices that isolate an aneurismal sac from the blood stream, following a minimally invasive surgical procedure.

According to an aspect of some embodiments of the present invention there is provided a medical device comprising at least one in vivo weldable polymeric component that is mounted on a balloon and that can be expanded in vivo. Additionally, according to an aspect of some embodiments of the present invention there is provided a medical device comprising at least one polymeric component that is mounted and wrapped or folded or wound around a balloon and that can be unwrapped or unfolded or unwound from the balloon, in vivo, and then, in some embodiments, also expanded, as required.

In some embodiments of the present invention, the polymeric device will expand without the need of an inflatable balloon, following the stimulation of one or more of several suitable triggers. Optionally, the in vivo weldable polymeric component may display Shape Memory, whereby, due to the stimulation of the trigger or triggers, will expand, become in tight contact with one or more in vivo weldable component/s, and the two or more components will become welded together in vivo. In some embodiments, the trigger may be present in vivo, such as, among others, the aqueous biological environment and/or physiological temperature, and combinations thereof, and/or the application of a trigger or triggers from outside the body. In some embodiments, said in vivo weldable polymeric component displaying Shape Memory may comprise a magnetic component that will be heated using an magnetic field, whereby said in vivo weldable polymeric component displaying Shape Memory heats up above the relevant temperature that will allow its transition to its previous shape. The magnetic component can be present throughout the in vivo weldable polymeric component displaying Shape Memory or in any selected region, such as on the surface, at its end, or any other distribution throughout the in vivo weldable polymeric component displaying Shape Memory. In some embodiments, said in vivo weldable polymeric component displaying Shape Memory may consist of more than one polymer displaying Shape Memory, differing in their properties, such as, without limitation, the conditions, such as, without limitation, the temperature and/or water content at which the transition takes place, their composition, their mechanical properties, being biodegradable or not, containing or not a drug or different drugs or any other material exhibiting any kind of biological or other activity or fulfilling any other kind of role/s, their distribution and spatial array in the polymeric component, their size, from nanometric to macroscopic, and any other property.

In some embodiments, an UltraSound field may perform as the heating trigger. In yet other embodiments, an exothermic chemical or biochemical reaction or a physical phenomenon may be perform as the source of heat, causing the in vivo weldable polymeric component displaying Shape Memory to go through the transition.

According to an aspect of some embodiments of the present invention there is provided a medical device comprising at least one metallic expandable tubular structure based on deploying the different components of the device sequentially and welding them together in vivo, often at their site of performance, namely in situ. The polymers able to perform successfully have not only to display enhanced mechanical properties but also with the specific biological requirements of each application. For example, in the case of blood-contacting devices, besides being able to soften and weld at slightly supra-physiological, acceptable temperatures (typically considered to be around or below 65° C.) or any alternative techniques, as disclosed hereby, they have to comply with the stringent hemo-compatibility requirements crucial to this indication.

In one embodiment of the present invention, the components of the device are sequentially mounted on a balloon, navigated to the aneurismal site one after another, brought into contact, softened—thermally or otherwise—and rapidly and tightly welded together, or welded to other components, to accessories or instruments or to tissues, and combinations thereof, as required by that specific application. If thermally, in one embodiment, a balloon filled with warm saline is used. The polymers used to generate part of the whole construct are tailored, therefore, to have a low softening temperature (typically in the 45 to 65° C. range). If thermally softened, after in vivo welding is achieved, the system subsequently cools down or is actively cooled down, whereby a very strong bond between the welded constituents is generated, and then the balloon is deflated and removed.

The novel in vivo weldability feature, unique to the devices disclosed hereby, allows constructing the devices in vivo or even in situ, namely, at their site of performance. In the case of a hybrid stent graft, the metallic stent and the polymeric conduit aqre deployed sequentially and welded together in vivo. By inserting the metallic stent and the polymeric component separately and welding them together at their site of performance, the size of each of the components is markedly smaller Moreover, in yet another embodiment of this invention, when required such in extreme situations requiring reducing the profile of the device even further, the polymeric component may be deployed using an “onion peel” strategy. In this case, ultra-thin layers of the in vivo weldable polymeric conduit will be deployed, one after another, with the first layer being welded to the stent, and the following ones, between them. As already stated, in one embodiment of this invention, the struts of the metallic stent are coated with an in vivo weldable material that will render the metallic stent in vivo weldable, and allow its in vivo welding to the in vivo weldable polymeric conduit. In other embodiments, the surface of the metallic struts is surface treated, so to render them with the ability to strongly connect to the polymeric conduit.

For the in vivo welding strategy to succeed, it has to comply with three key requirements: {a} it can be performed at a physiologically acceptable temperature; {b} a strong and long lasting welded connection is generated, and {c} the welded bond is rapidly formed.

Besides being of universal applicability, the in vivo welding concept will enable surgeons to overcome the three most frequently encountered and most challenging shortcomings limiting the use of stent graft devices presently in clinical practice:

(1) When the stent graft cannot be inserted due to narrow, tortuous or calcified access arteries.

(2) When modular overlapping components are deployed at branch points, such as the aorto-iliac bifurcation or branch arteries arising from the aorta, causing instability and blood leakage problems. and

(3) When anatomies that lack infra-renal landing zones require a safe, efficacious and “off the shelf” solution.

The in vivo welding capabilities engineered into these new devices will not only improve the quality of their performance, but will also significantly expand their clinical applicability. Moreover, patients that so far had no alternative but to undergo open surgery will now become eligible for implantation via ultra-minimal invasive procedures.

According to an aspect of some embodiments of the present invention there is provided a polymeric system configured to produce a polymeric material upon stimulation under physiological conditions, such that the stiffness of the polymeric material increases, preferably “on command”.

According to an aspect of some embodiments of the present invention there is provided a method of lining a body vessel, the method comprising introducing the medical device described herein into the lumen of a tissue or organ, such as, without limitation, a blood vessel, any section of the respiratory or urinary system, the GI tract, among numerous others.

According to an aspect of some embodiments of the present invention there is provided the use of a polymeric system having a stiffness which increases upon stimulation under physiological conditions in the manufacture of a medical device for the treatment of various pathologies such as, among numerous others, lining a body vessel and/or treating an aneurysm.

In several embodiments of the present invention, the stimulation upon which the in vivo polymeric component becomes stiffer optionally comprises cooling of the tubular structure from a temperature above body temperature, at which the thermoplastic polymer is sufficiently soft so as to render the component expandable, in addition to being in vivo weldable, to a body temperature (a physiological temperature). Optionally, the stimulation comprises a further type of stimulation in addition to the aforementioned cooling (e.g., any other type of stimulation described herein). Optionally, in some embodiments, the polymeric component displaying the stiffness differential described above, may not be in vivo weldable and may perform others roles.

It is to be appreciated that the stimulation by cooling may optionally comprise passive cooling, for example, by merely having the component in the body, without external heating, and allowing it to cool down to body temperature. Alternatively or additionally, the stimulation may comprise active cooling, for example, causing a fluid having a temperature below body temperature pass through the component (for example, without limitation, by means of passing such a fluid through an inflating balloon).

Utilizing an in vivo weldable device as disclosed in some embodiments of this invention, can be exemplified hereby when treating an aneurysm of a blood vessel by deploying a novel EVAR device, the novelty of whom is the ability of its components to be deployed sequentially and in vivo welded, whereby the final device is engineered, and is effected by:
introducing into the vessel the metallic stent and deploying it at its site of performance, the struts of said metallic stent being coated with an in vivo weldable polymer;
introducing into the vessel an in vivo weldable polymeric tubular structure made, at least in part, from an in vivo weldable polymer as described herein;
heating the in vivo weldable polymeric component using an inflatable balloon filled with suitably warm saline, so as to enable its easy expansion of the polymeric component and its attachment to the metallic stent;
welding both components together while the polymeric component is in intimate contact with the coated struts of the metallic stent, and under the suitable pressure applied by the warm balloon; and
generating conditions for the device to cool to a physiological temperature (e.g., 37° C.±5° C.), such that the metallic/polymeric device is strengthened and stabilized at its long term performance conditions, thereby lining the vessel and further thereby treating an aneurysm of a blood vessel.

Utilizing a thermoplastic polymer as described herein in the device described herein is associated with a thermal stimulation.

The thermal stimulation used for softening the in vivo weldable polymer can be effected by heating the polymer, for example, by means of placing a structure made from the polymer on a balloon filled with heated solution, as described herein. The heating can be applied prior to introducing the device into the vessel, in vivo or in situ, upon deployment of the structure. As noted hereinabove, the second thermal stimulation can comprise passively exposing the thermoplastic polymer to a physiological temperature (e.g., by arresting the heating) or by actively cooling the device containing the polymer in situ.

It is to be noted that a polymeric system used for forming a tubular structure as described herein, can comprise, according to these embodiments of the invention, in addition to a thermoplastic polymer, additional components, such that the stimulation(s) applied to such systems are manipulated accordingly.

As stated previously, in some instances, the in vivo weldable component may comprise a liquid, said liquid having the objective of rendering the polymeric component in vivo weldable, and/or increase its stiffness in due time, or impart any other property to the polymeric component. In some instances said liquid may be inert and unable of performing any chemical reaction, and in some embodiments it may undergo reactions of various types. One type of chemical reaction can be, for example, without limitation, to undergo degradation by different means, such as, without limitation, reacting with water molecules or enzymes. Another type can be, for example, without limitation, to be able to polymerize and or crosslink, due to the presence of moieties having the appropriate reactivity, number and availability. In some instances the liquid material is generated in vivo, following one or more mechanisms, such as, among others and without limitation, the liquid being the result of a chemical, physical or biological process, and combinations thereof. In some instances the liquid may be initially present within the polymeric component as a solid, of any size and/or geometry and/or distribution within the polymeric component and/or configuration, becoming a liquid in vivo, via a chemical, physical, mechanical or biological process, and combinations thereof. This can be illustrated, without limitation, by an embodiment where said solid is converted into a liquid by a solubilization process. The purpose of the liquid or the solid that will be converted into a liquid or part of it, is to form a dispersion or solution,. In some embodiments it may also change the local pH and/or ionic strength or any other property of that site and/or systemically. The systems are also referred to herein as “smart” systems, which include a “smart” component. For example, in several embodiments, the “smart” component is referred to herein as a substance that is responsive to a stimulation, such that its response results in a change in the ability of the polymeric component to be in vivo weldable, or in a change of its stiffness, among others, and combinations thereof. Other systems are also contemplated.

According to another aspect of some embodiments of the invention, there is provided a method suitable for lining a tissue or organ, such as, without limitation, a body vessel, the method comprising introducing the different components of the in vivo weldable medical device described herein into the vessel. Optionally, the vessel is any section of the respiratory system, or of the GI tract, or of the urinary system, or a blood vessel along the vasculature, or in the heart or brain, among numerous others. The device can be introduced via a minimally invasive procedure, preferably using a catheter or another delivery apparatus, as further detailed hereinabove.

The method optionally further comprises expanding or unfolding/unwrapping the different components of the in vivo weldable device in vivo, and in some embodiments in situ, to an expanded and/or unwrapped or unfolded state thereof. The diameter of the different components of the in vivo weldable device in its expanded and/or unwrapped or unfolded state is preferably the same or slightly larger than the diameter of the body vessel.

Optionally, the method further comprises decreasing the stiffness of the polymeric system of the device prior to the expansion of device, for example, by applying a suitable stimulation to the polymeric system. According to some embodiments, the method further comprises subjecting the device to a stimulation which increases the stiffness of the polymeric system, subsequent to the expansion of the device, as required and dictated by each clinical indication.

According to some exemplary embodiments, the different components of the in vivo weldable device are expanded using an inflatable balloon. Preferably, the different components of the in vivo weldable device can be mounted on the balloon prior to the delivery into the body. However, embodiments in which the balloon is delivered in its deflated state into the volume defined by the in vivo weldable component after the in vivo weldable component is in introduced into the body are not excluded from the scope of the present invention. The method optionally comprises inflating the balloon so as to expand the in vivo weldable component.

The balloon may optionally be a balloon designed and/or marketed for being inflated in a vessel of a living body. Such balloons will be familiar to a skilled practitioner (e.g., a surgeon). Representative examples include, without limitation, balloons employed in stent deployment procedures and the like.

In some embodiments of the present invention the different components of the in vivo weldable device comprises one or more in vivo weldable members, and the method comprises delivering the members separately into the site of deployment (sequentially using the same delivering device, or using different delivering devices which may optionally introduced into the site of deployment via different routes). Once the members are at the site of deployment the method preferably expands and/or unfolds and welds them in vivo to each other as further detailed hereinabove. Optionally, said members may be sequentially welded, or at any other order. In some embodiments, said members may be branched, or in a head-to-tail configuration or in a parallel configuration or in any other configuration and combinations thereof.

Optionally, the method comprises establishing fluid intercommunication between the lumens of the various members by forming in vivo an opening at the point of joining between the members as further detailed hereinabove.

In some embodiments of the present invention one or more of the different components of the in vivo weldable components forming the device is provided as two or more separate layer members, each constituted to form a layer of the respective component. In these embodiments, the method comprises sequentially deploying the layer members in vivo, preferably at the site of deployment, preferably, but not necessarily, using the same delivering device, to form, in vivo, a multilayer structure. Optionally, the method comprises welding the layers to each other as further detailed hereinabove.

The method may further comprise imaging at least the respective portion of the vessel in order to facilitate proper placement and expansion of the medical device. Examples of imaging techniques include, without limitation, visible light imaging, infrared imaging, magnetic resonance imaging, X-ray imaging, ultrasound imaging, and gamma ray and positron emission techniques. Imaging may be performed using a miniaturized imaging system mounted on a suitable catheter and introduced into the body, such as, without limitation, a body vessel, such as, without limitation, a blood vessel. In some embodiments of the present invention the miniaturized imaging system is mounted on the same catheter that is used for delivering the device to the site of deployment. Alternatively or additionally, external imaging may be employed. Imaging may be performed with the aid of diagnostic agents. The most beneficial use of imaging in the context of the present invention is expected to be addition to the blood vessel of the patient of a diagnostic agent such as a contrast agent, in order to present an image of the blood vessel while introducing the medical device into the blood vessel.

The term “lining”, as used herein, describes the process of covering a section of the inside wall of a tissue or organ, such as, without limitation, a vessel, such as, without limitation, a blood vessel, with at least one layer of material, without significantly impeding flow of a fluid (e.g., blood flow).

Lining a blood vessel may be intended, without limitation, for treating a hole in the vessel wall (e.g., a hemorrhage, a wound, a ruptured aneurysm) by covering the hole, optionally releasing beneficial drugs (e.g., drugs incorporated in the medical device and/or the polymeric system), inhibiting or reducing turbulent blood flow, treating atheromatous plaques and/or blood clots (e.g., by isolating the plaque debris and/or blood clots and/or trapping them so as to prevent or reduce the release of these plaque debris and/or blood clots into the bloodstream).

In some exemplary embodiments, lining a blood vessel is for treating an aneurysm in the blood vessel in a subject in need thereof. For example, the device may isolate the aneurysmal sac from the blood flow, thereby improving blood flow, reducing the likelihood of aneurysm rupture, and/or minimizing the lethality of aneurysm rupture.

Optionally, the aneurysm is an aortic aneurysm (e.g., an abdominal aortic aneurysm, a thoracic aortic aneurysm).

The methodologies disclosed herein are effective at lining a vessel and treating an aneurysm in a controllable and predictable manner Unlike the traditional EVAR methodologies, the methodologies disclosed herein in accordance with some embodiments of the invention are of a relatively simple construction, are not very costly, and are preferably devoid of the drawback characteristic of the EVAR currently in clinical use. The present EVAR systems consist of a fabric and a metallic mesh, connected together, typically by sewing. The invention disclosed hereby teaches to generate the EVAR in vivo, by deploying its different components separately, typically, first the metallic stent, made in vivo weldable by adding an in vivo weldable polymer to it, and then the in vivo weldable polymeric component, that will be welded to the metallic stent in vivo. By performing the procedure using the devices disclosed hereby and the teachings taught by this invention, the size of the pathway required to access the site of performance may be significantly smaller, and also, since each of the components is also significantly more flexible that the stent/fabric complex used is EVARs of the prior art, the degree of tortuosity of the pathway of access can be higher, without impeding the deployment of the components.

It is therefore believed that in accordance with some of its embodiments, the present invention represents an improvement over current EVAR technology.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 DSC thermogram of a welding product of an elastomeric polyurethane (CLUR) and a stiff polymethacrylate (PEMA).

FIGS. 2A-F exemplify a welding method accoridng to the invention using a transparent Tygon model system and a tubular thin device with white holes, shown in FIG. 2A. In FIG. 2B, the device is inserted within the “Tygon vessel”, and in FIG. 2C this first layer of the device is easily expanded and attached to the luminal surface of the “vessel”. In FIG. 2D, the balloon is deflated and removed, while FIGS. 2E and 2F, demonstarte the deployment of a second layer, white and longer, and its subsequent welding together to the first layer already deployed and expanded at the site of performance.

FIGS. 3A-F provide photos of an exemplary system according to the invention constructed of two different metallic structures, one flat (FIGS. 3A-C) and one tubular (FIGS. 3D-F), demonstrating the in vivo welding concept.

FIG. 4 shows a model system, mimicking the anatomy of aorta and one renal artery.

FIG. 5 illustrates another embodiment of the invention.

FIGS. 6A-B demonstrate a stent according to the invention, wherein in FIG. 6A a coating of a strut of a stent with an in vivo weldable polymer is shown under amplification, and in FIG. 6B the thickness of the coating is shown.

FIG. 7 shows welding of an in vivo weldable patch.

FIGS. 8A-D follow a measurement of force required to pull out a patch, welded to a stent (FIGS. 8A-B), using a tensiometer (FIG. 8C). FIG. 8D shows the measured load.

FIGS. 9A-C show manual extension of a patch welded to the stent.

FIG. 10 shows the last stage of a mechanical testing of the strength of the welding bond between the stent and the patch.

FIG. 11 provides a SEM micrograph of a coated stent strut and its welding to an in vivo polymeric patch.

### DETAILED DESCRIPTION OF EMBODIMENTS

### EXAMPLES

Reference is now made to the following examples, which together with the above descriptions illustrate some embodiments of the invention in a non limiting fashion.

### Materials and Methods

### Materials:

Benzoyl peroxide was obtained from Fluka.
c-Caprolactone was obtained from ACROS Organics.
Ethylene glycol dimethacrylate (EGDMA) was obtained from Aldrich.
Hexamethylene diisocyanate (HDI) was obtained from Aldrich.
2-Hydroxyethyl methacrylate (HEMA) was obtained from Aldrich.
L-lactide and D-lactide were obtained from Boehringer Ingelheim.
D,L-lactide was obtained from ACROS Organics.
N,N-dimethyl-p-toluidine was obtained from Aldrich.
Polyacrylic acid (PAA) was obtained from Aldrich.
Polycaprolactone (PCL2000, PCL1250 and PCL530) was obtained from Aldrich.
Polyethylene glycol 2000 (PEG2000) was obtained from Aldrich.
Polyethylene glycol 6000 (PEG2000) was obtained from Merck.
Polymethyl methacrylate (PMMA) was obtained from Aldrich.
Poly(styrene-methyl methacrylate) (SMMA) was obtained from Aldrich.
Polytetramethylene glycol (PTMG650 and PTMG1000) was obtained from Aldrich.
Stannous 2-ethyl hexanoate was obtained from Sigma.

### Methods:

Polymer molecular weights were characterized by Gel Permation Chromatography (GPC), using a Waters 2690 Separation Module with a Waters 410 Differential Refractometer and Millenium Chromatography Manager.

Thermal properties and crystallinity were characterized using a Mettler TA 3000 Differential Scanning calorimeter.

Mechanical properties were determined using an Instron apparatus.

### Example 1

### Polymer Syntheses

### Polycaprolactone Polyurethane (CLUR)

Polycaprolactone polyurethane co-polymers are generally prepared by co-polymerizing a polycaprolactone-based polymer with hexamethylene diisocyanate, following the exemplary procedures described hereinafter. The polycaprolactone chain is terminated with functional groups that will allow it to react with the diisocyanate, for example, hydroxy, amine, thiol or carboxylic acid groups.

The polycaprolactone-based polymers PCL2000, PCL1250 and PCL530 were copolymerized with hexamethylene diisocyanate (HDI) to obtain copolymers referred to herein as CLUR (caprolactone urethane) polymers.

As an example, the synthesis of CLUR2000 from PCL2000 and HDI is described in detail as follows.

50.0 grams of OH-terminated PCL2000 was dried at 120° C. under a vacuum for 2 hours with magnetic stirring. Hexamethylene diisocyanate and stannous 2-ethyl hexanoate were added to the reaction mixture at molar ratios of 1:1 (to PCL2000) and 1:100 (to PCL2000), respectively, and reacted at 90° C. for 30 minutes with mechanical stirring under a dry nitrogen atmosphere. The obtained product was dissolved in 150 ml dry dioxane and precipitated in 1200 ml petroleum ether 40-60. The polymer referred to herein as CLUR2000, was then filtered and dried under a vacuum at room temperature for 24 hours.

Using essentially the same procedures, various CLUR polymers were prepared using PCL diol segments, from PCL530 to PCL 12000.

The molecular weights, polydispersity indices (PDI), thermal properties and mechanical properties of a few of the CLUR polymers are shown in Table 1.

TABLE 1
Properties of CLUR polymers

| | | | | | Crystal- | |
|---|---|---|---|---|---|---|
| | Mn | MW | | Tm | linity | Modulus |
| Polymer | (g/mol) | (g/mol) | PDI | (° C.) | (%) | (MPa) |
| CLUR530 | 126,200 | 137,300 | 1.1 | 40 | 11 | 48 ± 8 |
| CLUR1250 | 98,400 | 111,500 | 1.1 | 38 | 16 | 32 ± 6 |
| CLUR2000 | 107,400 | 121,400 | 1.1 | 48 | 26 | 183 ± 6 |
| CLUR530- | 120,000 | 130,500 | 1.1 | 36 | 14 | 14 ± 3 |
| 2000 1:1 | | | | | | |

In comparison, PCL2000 exhibited a Tm of 59° C. and a crystallinity of 51%.

CLUR2000 exhibited water absorption of 3-5%.

Poly(caprolactone-ethylene glycol) polyurethane (e-CLUR):

Poly(caprolactone-ethylene glycol) polyurethane co-polymers are generally prepared by co-polymerizing a polycaprolactone-based polymer with a polyethylene glycol and hexamethylene diisocyanate, following the exemplary procedures described hereinafter. The preparation follows a similar chemistry as the preparation of CLUR polymers described above.

Polycaprolactone-based polymers were copolymerized with polyethylene glycol (2 kDa or 6 kDa) and hexamethylene diisocyanate (HDI), to obtain copolymers referred to herein as e-CLUR polymers.

e-CLUR2000 was prepared using various PEG:PCL molar ratios. The synthesis of e-CLUR2000 with a 1:10 PEG:PCL molar ratio is described in detail as follows.

25.0 grams of OH-terminated PCL2000 and 2.5 grams of PEG2000 were dried at 120° C. under a vacuum for 2 hours with magnetic stirring. Hexamethylene diisocyanate (HDI) and stannous 2-ethyl hexanoate were then added to the reaction mixture at molar ratios of 1:1 and 1:100 (to total PCL2000 +PEG2000), respectively, and reacted at 90° C. for 30 minutes with mechanical stirring under a dry nitrogen atmosphere. The obtained product was dissolved in 150 ml dry dioxane and precipitated in 1200 ml petroleum ether 40-60. The e-CLUR2000 polymer was then filtered and dried under a vacuum at room temperature for 24 hours.

Using essentially the same procedures, e-CLUR2000 (e-CLUR2K-2K) was prepared using 2:10 or 3:10 PEG:PCL molar ratios. In addition, e-CLUR2K-6K was prepared using PCL2000 and PEG6000.

The molecular weights, polydispersity indices (PDI), thermal properties and mechanical properties of the e-CLUR polymers are shown in Table 2.

TABLE 2
Properties of e-CLUR polymers

| Polymer | | | | | | |
|---|---|---|---|---|---|---|
| (molar % of PEG relative | Mn | MW | | Tm | Crystallinity | |
| to PCL) | (g/mol) | (g/mol) | PDI | (° C.) | (%) | Modulus (MPa) |
| e-CLUR2K-2K (10%) | 86,000 | 103,900 | 1.2 | 46 | 27 | 194 ± 17 |
| e-CLUR2K-2K (20%) | 98,900 | 112,600 | 1.1 | 48 | 26 | 187 ± 14 |
| e-CLUR2K-2K (30%) | 90,400 | 106.600 | 1.2 | 47 | 21 | 147 ± 6 |
| e-CLUR2K-6K (10%) | 124,600 | 180,400 | 1.4 | 55 | 29 | 191 ± 15 |

e-CLUR polymers were considerable more hydrophilic than CLUR polymers, with e-CLUR2000 exhibiting water absorption of 120-150%, in contrast to the 3-5% absorption by CLUR2000.

### Polytetramethylene Glycol (PTMG) Polyurethane:

Polytetramehylene glycol polyurethane co-polymers are generally prepared by co-polymerizing polytetramethylene glycol with a bifunctional molecule, such as a diisocyanate (e.g., hexamethylene diisocyanate), following the exemplary procedures described hereinafter.

20.0 grams of PTMG650 were dried at 120° C. under a vacuum for 2 hours with magnetic stirring. Hexamethylene diisocyanate (HDI) and stannous 2-ethyl hexanoate were then added to the reaction mixture at molar ratios of 1:1 and 1:100 (to PTMG650), respectively, and reacted at 70° C. for 1 minute, with mechanical stirring under a dry nitrogen atmosphere. The obtained product was dissolved in 150 ml dry dioxane and precipitated in 1200 ml petroleum ether 40-60. The PTMG polyether-urethane polymer was then filtered and dried under a vacuum at room temperature for 24 hours.

The modulus of the PTMG650 polyurethane was 90±8 MPa.

Poly(caprolactone-lactic acid) polyurethane:

Poly(caprolactone-lactic acid) polyurethane co-polymers are generally prepared by co-polymerizing a polycaprolactone-based polymer with lactides (e.g., L-lactide, D-lactide or D,L-lactides) and hexamethylene diisocyanate, following the exemplary procedures described hereinafter.

Triblock copolymerss of polylactic acid-polycaprolactone-polylactic acid (PLA-PCL-PLA) were prepared by the ring opening polymerization of the lactide (L-lactide, D-lactide or D,L-lactides) initiated by the hydroxyl end groups of the PCL polymers. Chain extension of the triblock was then carried out using hexamethylene diisocynate (HDI), producing a polyester-urethane.

Poly(caprolactone-tetramethylene glycol) (PCL-PTMG) polyurethane:

Poly(caprolactone-tetramethylene glycol) polyurethane co-polymers are generally prepared by co-polymerizing a polycaprolactone-based polymer with polytetramethylene glycol and hexamethylene diisocyanate, following the exemplary procedures described hereinafter.

Triblock copolymers of polycaprolactone-polytetramethylene glycol-polycaprolactone (PCL-PTMG-PCL) are prepared by the ring opening polymerization of c-caprolactone initiated by the hydroxyl end groups of polytetramethylene glycol (e.g., PTMG1000). Chain extension of the triblock is then carried out using hexamethylene diisocyanate (HDI), producing a polyether-ester-urethane.

### Example 2

### Preparation of In Vivo Weldable Polymeric Components

The following describes exemplary methodologies used for preparing a device according some embodiments of the invention.

### Dip Coating:

Devices were prepared by dip coating on a suitable mold, typically a cylindrical (4-10 mm diameter) polytetrafluoroethylene-coated mandrel, by slowly dipping the mold into a container containing a solution of 15-20% (w/w) polymer in chloroform, and then slowly withdrawing the mold.

Dipping and withdrawing the mold was performed at a constant velocity in order to obtain a uniform coating. An electronic motor was used to control the vertical movement and speed during the dipping and withdrawing of the mold. The polytetrafluoroethylene-coated mandrel was dipped 7 cm into the polymer solution, typically using a cross head speed (CHS) of 10 mm per minute.

For the formation of devices with a wall thickness of 100-700 μm, 3 to 10 dipping cycles were preformed, and the polytetrafluoroethylene-coated mandrel was then dried at room temperature overnight. After the evaporation was complete and the polymer was dry, the polymer tube was extracted from the mandrel.

CLUR2000 and e-CLUR2000 tubes prepared according to this method are shown below.

### Electrospinning:

Electrospinning is a technique capable of producing nanometric fibers in a relatively well controlled and reproducible manner, producing highly porous 2-dimensional meshes as well as 3-dimensional constructs. Electrospinning is performed by applying a high voltage, using an electrode, to a capillary filled with the polymer fluid to be spun. The resulting fibers are collected on a grounded plate.

In an exemplary procedure, 8-15% (w/w) polymer solutions in chloroform were used, and the grounded plate was metal mandrel with a 5.5 mm diameter. The distance between the electrospinning needle and the collector mandrel was between 10-60 cm, depending on the thickness of the fibers to be obtained. Voltages in a range of from 5 kV to 30 kV were utilized for the formation of device walls with thicknesses in a range of from 100 μm and 700 μm, respectively.

### Air Spray:

This technique is capable of forming nanometric and micrometric fibers in a relatively well controlled and reproducible manner, producing porous structures. The air spray technique is conducted by passing high pressure dry air through a capillary filled with a solution containing the polymer to form an aerosol, which is sprayed on a collector, such as a rotating polytetrafluorethylene-coated mandrel. In an exemplary procedure, 8-15% (w/w) polymer solutions in chloroform were used. The distance between the polymer spray gun and the collector mandrel varied between 10 cm and 60 cm, for the formation of the devices with wall thicknesses ranging from 100 μm to 700 μm. A 2 bar air pressure was applied.

The air spray technique produces a polymer in the form of a network of fibers.

The diameter of the fibers in the network depends on the type of polymer, its molecular weight, the concentration of the polymer in the aerosol solution, the solvent and the distance between the spray gun and the mandrel.

### Example 3

### Expanded In Vivo Weldable Polymeric Components

In vivo weldable polymeric components prepared from CLUR2000 using the air spray technique described above were expanded by inserting a balloon into the in vivo weldable polymeric component and inflating the balloon with warm (50° C.) water.

Due to the shape of the balloon, the tubular structures were expanded primarily in their mid-section. The less expanded edges of the tubular structures were cut off in order to better observe the expanded middle sections. The diameter of the tubular CLUR2000 structures could be increased considerably by expansion.

Additional air-sprayed CLUR2000 tubular structures were expanded as described above using a balloon which expanded the full length of the tubular structures. The dimensional changes of tubular structures as a result of expansion were then measured and are given in Table 3 below.

TABLE 3
Dimensional changes of tubular structures as a result of expansion

| Dimension | Before expansion | After expansion | Change |
|---|---|---|---|
| Length (cm) | 7.5 | 7.5 | +0% |
| Inner diameter (mm) | 5.3 | 14.1 | +266% |
| Outer diameter (mm) | 8.2 | 15.3 | +186% |
| Thickness (mm) | 1.4 | 0.6 | −60% |

The effect of expansion on the stiffness of the tubular structures was measured by determining the transverse moduli of the structures before and after expansion.

The mechanical properties of the tubular structure were also determined before and after expansion. The expansion described above increased the modulus from 26±2 MPa to 82±9 MPa, the strain at peak was reduced by expansion from 285±42% to 28±4%, and the stress at peak was increased by expansion from 4.9±0.2 MPa to 9.0±0.6 MPa.

Furthermore, the transition temperature was essentially unchanged by expansion, whereas the crystallinity of the polymer in the tubular structure increased from 26.12% before expansion to 31.59% after expansion.

Expanded tubular structures were re-warmed by reinserting the balloon into the lumen of the structure and filling the ballon with warm (50° C.) water. The balloon was then deflated by removal of the water at a rate of 0.25 ml/second. The tubular structure contracted as the balloon deflated, and the inner wall of the tubular structure remained attached to the balloon.

The expansion and contraction of the tubular structures were reversible over the course of at least 3 or 4 cycles of expansion and contraction.

### Example 4

Polymer In Vivo Weldable Polymeric Components with an Adhesive Coating

A biocompatible adhesive substance in solid (e.g., powder), semisolid (e.g., gel) or liquid (e.g., solution) form is added to the outer surface of a polymer device, to produce an adhesive coating. The adhesive substance may be added as a layer on top of the outer surface of the polymer device or as a layer incorporated into the polymer of the polymer device.

In an exemplary procedure, biocompatible polyacrylic acid adhesive coatings were added to polymer devices prepared as described hereinabove, according to the following exemplary procedures.

A 2.5% solution of polyacrylic acid (typically having a molecular weight of 1,250,000) in ethanol is sprayed on the top of the outer layer of the device using the air spray technique described above. The device is then dried in a vacuum at room temperature in order to remove all traces of the solvent.

In an alternative method, powdered polyacrylic acid is homogeneously dispersed on the outer layer of the device. An additional thin layer of fibers is then sprayed over the polyacrylic acid particles in order to retain them on the outer surface of the device.

When the device is exposed to the biological aqueous environment, the polyacrylic acid coating becomes adhesive, which improves the ability of the device to adhere to tissue and remain in place.

### Example 5

In Vivo Weldable Polymeric Components with Polyurethane Foam

Compressible cuffs are prepared from a foam comprising an elastomer (e.g., a polyurethane and/or a silicone elastomer) and attached (e.g., by crimping) to an outer surface of a polymeric component. The cuffs may cover the outer surface of the whole component or cover the ends of the device or following any other pattern.

In an exemplary procedure, highly compressible (95% compression) polyurethane foam cuffs were attached to the ends of an in vivo weldable polymeric component prepared as described hereinabove. The foam cuffs were attached by placing the cuffs around the edges of the in vivo weldable polymeric component and then crimping the edges of the in vivo weldable polymeric component, as shown below. The foam cuffs are for improving the ability of the device to grip to a surface, in specific embodiments.

### Example 6

### In Vivo Weldable Polymeric Components in a Branched In Vitro Aorta-Renal Ranch Model

This example aims at showing the ability of the polymeric components of this invention to easily, rapidly and strongly in vivo weld, under moderate heating and pressure, so they can be used for both shaping and welding a device in situ.

An in vivo weldable branched polymeric component was tested using an in vitro model of the aorta-renal branch, which was constructed from perpendicular polymeric tubes. The branched polymeric component was constructed in vivo from two in vivo weldable tubular structures prepared from CLUR2000 using the air-spray technique described above.

In the first step, an in vivo weldable polymeric component was deployed in the smaller tube of the in vitro model, which corresponds to the renal artery, and said in vivo weldable polymeric component had, in a specific embodiment, a tubular structure with an expanded annular area at the proximal end, namely that that faces the aorta. The branch component was placed in the tube of the in vitro model which corresponds to the renal artery, with the expanded annular area slightly protruding into the tube corresponding to the aorta. The purpose of this expanded annular area of the in vivo weldable component deployed in the renal artery is to generate a larger are of welding with the in vivo weldable component to be deployed in the aortic vessel, as described below.

The component was then expanded “in situ” by inserting a balloon into its lumen and inflating the balloon with warm (typically around 50° C.) water, until the branch component attached firmly to the walls of the “renal artery”. The balloon was also placed in the “aorta” adjacent to “renal artery”, and inflated with warm water until the expanded annular area of the branch component tightly attached to the wall of the “aorta”.

In the second step, a main in vivo weldable component was deployed in the tube of the in vitro model which corresponds to the aorta, perpendicularly to the previously deployed branch component, as described above.

The main in vivo weldable component is then expanded and welded together “in situ” to the in vivo weldable branch component previously deployed within the “renal artery”, by inserting a balloon into the main component and inflating the balloon with warm (typically around 50° C.) water until the pressure required, typically of at least 2 atmospheres, is achieved.

Then, a hole was then formed outwardly in the wall of the main in vivo weldable component so as to form a single branched structure comprising the two welded components, such that a fluid may flow freely from one component to the other. The balloon was further inflated with warm water in the area of the hole so as to cause protrusions and flaps created by formation of the hole to weld to the internal wall of the branch component. In some embodiments, said hole is preformed, and not generated in vivo.

The model was then dissected and the branched component was removed and analyzed. The two tubular components had been welded together, and the inner surfaces of the tubular structures were smooth, showing that protrusions and flaps formed by creation of the hole were fully welded to the walls of the branch component. The two components, namely the main and the branch in vivo weldable components were strongly welded by both the expanded annular section of the branch, that was welded to the external wall of the main component, and the protrusions and flaps generated by the hole, that welded into the internal wall of the branch component.

These results indicate that moderate heating can be used for both shaping and welding a device in situ.

### Example 7

### In Vivo Weldable Polymeric Components in Cadaveric Pig Aorta Sections

This example aims at showing the ability of the polymeric components of this invention to easily, rapidly and strongly expand under moderate heating and pressure, so they can be used for both shaping and welding a device in vivo. Even though this example does not relate to the in vivo welding of the component, the expandability is a key feature of some of these devices, so they can be brought in contact with another in vivo weldable component and welded together. Among many others, for example the struts of a metallic stent, coated with an in vivo weldable polymer.

An in vivo weldable polymeric component prepared from CLUR2000 by air spray, as described hereinabove, was tested in cadaveric pig aorta section. The in vivo weldable polymeric component was expanded in situ with a balloon filled with warm (50° C.) saline, until the in vivo weldable polymeric component tightly and securely adhered to the walls of the aorta. The attachment of the component to the walls of the aorta lumen was then assessed.

After 8 hours, the pig was sacrificed, and the aorta was examined The diameter of the polymeric component increased by a factor of more than 3, and it became tightly attached to the luminal surface of the vessel. The placement was secure, as it was extremely difficult to remove the polymeric component from the aorta section, following explantation. The force required to remove the polymeric component from the aorta section, was approximately 10 times the force typically applied by blood flow at this site.

### Example 8

### An In Vivo Weldable Polymeric Component Deployed in an Ex Vivo Model

This example aims at showing the ability of the polymeric components of this invention to easily, rapidly and strongly expand under moderate heating and pressure in an in vivo model. Even though this example does not relate to the in vivo welding of the component, the expandability is a key feature of some of the embodiments of the invention disclosed hereby, so they can be brought in contact with another in vivo weldable component and welded together. Among many others, for example, the struts of a metallic stent, coated with an in vivo weldable polymer.

An in vivo weldable polymeric component prepared from CLUR2000 by air spray, as described hereinabove, was mounted on a balloon and deployed ex vivo by inflating the balloon in situ with warm water. The polymeric component had excellent mechanical properties so as to maintain its shape after deployment in the vessel and also “pulsate” in unison with the vessel, when an external force was applied. The placement was secure, and it was extremely difficult to remove the expanded in vivo weldable polymeric component from the aorta. In a pull out test, the force required to remove the expanded polymeric component was around 45 N.

### Example 9

### Sealing of an Aneurysm in an In Vitro Model

An in vitro model of an aneurysm was used to determine the ability of an in vivo weldable polymeric component according to embodiments of the invention to seal an aneurysm and improve blood flow. The aneurysm model was prepared from latex, by dip coating a metal mold in a latex solution, and then drying the latex layer and removing the mold.

An in vivo weldable polymeric component with foam cuffs was prepared as described above and placed in the aneurysm model.

The in vivo weldable polymeric component walls proved to be impermeable to liquid, such that liquid passed through it without leaking into the aneurismal sac.

Moreover, vacuum could be applied to the aneurysm, indicating that the endograft sealed the aneurysm against gases, in addition to liquids.

### Example 10

In Vivo Weldable Polymeric Component with a “smart” Monomer Component

A polymer is mixed with a monomer which can be polymerized by a suitable stimulation, such that the mixture is an expandable polymeric component. The monomer per se softens the polymer (e.g., by acting as a plasticizer), whereas the polymerized monomer is a solid material which provides mechanical support, and consequently strength and stiffness, to the polymer which was originally in the system. The monomer is thus a “smart component” for softening and then hardening the polymeric component, as required and when desired.

In an exemplary procedure, films containing various mixtures of a polymer and a monomer were prepared.

PMMA, HEMA and benzoyl peroxide (BP) were dissolved in chloroform at various PMMA:HEMA ratios, and with 100:1 HEMA:BP ratio (w/w). The solution was cast in a Petri dish and the chloroform was allowed to evaporate during the course of 24 hours. Dog-bone samples were cut out of the obtained film, and their modulus was measured using an Instron apparatus. HEMA was then polymerized within the PMMA matrix by adding N,N-dimethyl-p-toluidine to the surface of the PMMA/HEMA films and then incubating the film for 1 hour at 37° C. The modulus of the reacted samples was measured using an Instron apparatus.

As shown below, HEMA considerably reduced the moduli of HEMA:PMMA mixtures in a concentration-dependent manner. As further shown therein, polymerization of the HEMA considerably increased the moduli of the mixtures.

Films containing poly(styrene-methyl methacrylate) (SMMA) and HEMA were prepared as described above for PMMA/HEMA films.

As shown below, HEMA considerably reduced the moduli of HEMA:SMMA mixtures in a concentration-dependent manner, as for HEMA:PMMA mixtures.

The glass transition temperatures (T_{g}) of the HEMA:SMMA mixtures were determined by Differential Scanning calorimetry.

As shown below, the glass transition temperatures of SMMA decreased considerably in the presence of HEMA. The decrease was concentration-dependent, with 10-30% HEMA resulting in a transition temperature in a range of about 40-55° C., in contrast to the 100° C. transition temperature in the absence of HEMA.

In addition, films containing CLUR2000 as an expandable component (EC) and HEMA as a smart component (SC) were prepared as described above for PMMA/HEMA films.

As shown below, HEMA considerably reduced the moduli of CLUR2000 mixtures in a concentration-dependent manner. As further shown therein, polymerization of the HEMA considerably increased the moduli of the mixtures.

As is further shown, the moduli of CLUR2000 and CLUR2000/HEMA mixtures were significantly lower than the moduli of PMMA, SMMA and the corresponding PMMA/HEMA and SMMA/HEMA mixtures.

In addition, films containing 80 kDa polycaprolactone (PCL80K) as an expandable component and ethylene glycol dimethacrylate (EGDMA) as a smart component were prepared as described above for PMMA/HEMA films.

As shown, EGDMA considerably reduced the moduli of PCL8OK mixtures in a concentration-dependent manner

The above results indicate that various monomers can be used as smart components for both softening polymeric materials to varying degrees and hardening the material when desired by polymerization of the smart component.

### Example 11

In Vivo Weldable Polymeric Component with a Cross-Linking “Smart” Component

An in vivo weldable polymeric component is prepared by using an expandable polymeric material comprising a functional group (e.g., thiohydroxy, amine, azide, alkyne, an unsaturated bond, a nucleophilic leaving group) and a cross-linking molecule, such as an at least bi-functional molecule (e.g., a diacrylate, a dimethacrylate, a dithiol, a diamine), which comprises functional groups (e.g, a nucleophilic leaving groups, unsaturated bonds, alkyne groups, azide groups, thiohydroxy groups, amine groups) capable of reacting with the functional group of the polymeric material. For example, alkyne groups may be reacted with azide groups by click chemistry.

Under physiological conditions and/or a suitable trigger, the reactions between the cross-linking molecule and the polymeric component are initiated, resulting gradually in cross-linking of the polymeric material. The modulus of elasticity of the polymeric component gradually increases over a period of time as the amount of cross-links increases, and the device becomes stronger and stiffer, such that the expanded state of the device is maintained.

In an exemplary procedure, a polymer (e.g., poly(2-hydroxyethyl methacrylate)) comprising alkyne groups (e.g., by linking propargyl alcohol to the polymer via hexamethylene diisocyanate, as show below), is reacted in vivo with a cross-linking molecule comprising azide groups (e.g., polyoxyethylene bis(azide)) via copper(I) catalysis, as shown below. The copper-catalyzed “click” reaction between the azide and alkyne groups results in a cross-linked polymer, which causes the structure to become stiffer.

### Example 12

“Smart” In Vivo Weldable Polymeric Component with Cross-Linking Functional Groups

An in vivo polymeric component is prepared comprising an expandable polymeric system comprising two complementary functional groups (e.g., an azide and an alkyne, unsaturated carbon-carbon bond and a thiohydroxy, an unsaturated carbon-carbon bond and an amine, a carboxylic acid and an amine, a hydroxy and an isocyanate, an amine and an isocyanate, and a thiohydroxy and an isocyanate) attached to a polymer (e.g., as substituents attached to the polymer backbone). The polymeric component may comprise a polymer having two complementary functional groups, or two polymers, each having a functional group complementary to the functional group of the other polymer.

Under physiological conditions or due to the application of a trigger, reactions between the complementary functional groups are initiated, resulting gradually in cross-linking of the polymer molecules in the polymeric system. The modulus of elasticity of the polymeric system gradually increases over a period of time as the amount of cross-links increases, and the device becomes stronger and stiffer, such that the expanded state of the device is maintained.

In an exemplary procedure, a polymer comprising an azide group is prepared using a monomer (e.g., 2-hydroxyethyl methacrylate) with an azide-containing monomer, 2-(2-azidoisobutyloxy)ethyl methacrylate and an alkyne-containing monomer.

The azide-containing monomer is prepared by first preparing 2-(2-bromoisobutyloxy)ethyl methacrylate [Xu et al., J Poly Sci A: Poly Chem. 46, 5263-5277 (2008)] by reacting 2-hydroxyethyl methacrylate with 2-bromoisobutyl bromide, and then reacting the 2-(2-bromoisobutyloxy)ethyl methacrylate with sodium azide.

The alkyne-containing monomer is prepared by linking propargyl alcohol to a monomer (e.g., 2-hydroxyethyl methacrylate) via hexamethylene diisocyanate, similarly to the method described below.

The azide-containing monomer is then copolymerized with the alkyne-containing monomer (e.g., by free radical polymerization), with or without an additional monomer such as 2-hydroxyethyl methacrylate, to obtain a polymer having both azide and alkyne groups.

A device comprising the obtained polymer is placed in a vessel in a body, and the polymer is reacted in situ by copper(I) catalysis. The copper-catalyzed “click” reaction between the azide and alkyne groups results in a cross-linked polymer, which causes the device to become stiffer.

In an additional exemplary procedure, a polymer comprising an azide group is prepared by polymerizing (e.g., by free radical polymerization) the azide-containing monomer described hereinabove, with or without copolymerization with additional monomer such as 2-hydroxyethyl methacrylate. In addition, a polymer comprising an alkyne group is prepared by polymerizing (e.g., by free radical polymerization) the alkyne-containing monomer described hereinabove, with or without copolymerization with additional monomer such as 2-hydroxyethyl methacrylate.

A device comprising the polymer comprising an azide group and the polymer comprising an alkyne group is placed in a vessel in a body, and the two polymers are reacted in situ by copper(I) catalysis. The copper-catalyzed “click” reaction between the azide and alkyne groups results in cross-linking of the two polymers, which causes the device to become stiffer.

Click chemistry encompases several reactions that are fast, selective, high yielding, and can be conducted in aqueous media and aerobic systems. The most common of these efficient reactions is the copper-catalyzed azide-alkyne cycloaddition, but the toxicity of copper led to the development of bio-orthogonal reactions whose components are inert to the surrounding biological environment and lack metal catalysts, called Cu-free click reactions. One important type of Cu-free click chemistry is the reaction between azide groups and strained cyclo-octyne moieties. One embodiment of the invention harnesses this chemistry to in situ react two polymers, whereby a substantial stiffening of said polymeric system takes place once the tubular member has been deployed and expanded at the site of an aneurismal sac. One example of this embodiment is the reaction of a derivatized poly(acrylic acid), comprising pendant azide groups, and a derivative of poly(hydroxyl ethylmethacrylate) having pendant cyclo-octyne groups, as follows:

### Synthesis of Cyclo-Octyne-Containing Polymer:

8,8-Dibromobicyclo[5.1.0]octane was synthesized according to procedure for the synthesis of 9,9-dibromo[6.1.0]nonane. Then, it was reacted with polyhydroxyethylmethacrylate, AgClO₄ and MeNO₂ to obtain poly((Z)-2-bromocyclooct-2-enyloxyethylmethacrylate). The product was converted into poly(Cyclooct-2-ynyloxyethylmethacrylate) through a two step reaction with (1) 1,8-diazabicyclo[5.4.0]undec-7-ene and (2)NaOMe and water.

### Synthesis of Azide-Containing Polymer:

Polyacrylic acid was reacted with thionyl chloride and O-(2-Aminoethyl)-O′-(2-azidoethyl)nonaethylene glycol was added to produce an amide derivative of polyacrylic acid with pendant azide groups. Alternatively O-(2-Aminoethyl)-O′-(2-azidoethyl)monaethylene was reacted with mehtylene chloride and then polymerized with CuBr/2,2-bipyridine to obtain the same product.

The two polymers are subjected to conditions that affect a Cu-free click reaction.

### Example 13

In Vivo Weldable Polymeric Component with a Plasticizer “Smart” Component

An in vivo weldable polymeric component is prepared comprising an expandable polymeric system comprising a polymer and a small hydrophilic molecule, such as low-molecular weight (e.g., 250-850 grams/mol) polyethylene glycol which plasticizes the polymeric material, thereby rendering the component more expandable and less stiff.

Continuous contact with an aqueous environment in vivo results in gradual leaching of the hydrophilic plasticizer from the device. The modulus of elasticity of the polymeric system gradually increases over a period of time as the concentration of plasticizer decreases, and the device becomes stiffer, such that the expanded state of the device is maintained.

### Example 14

### In Vivo Weldable Polymeric Components Comprising “Smart” Amorphous Polymers

An in vivo weldable polymeric component is prepared comprising an amorphous polymer capable of undergoing considerably morphological changes by crystallization, resulting in a pronounced increase in the strength and stiffness of the material. The polymeric component is deployed and expanded in its non-crystalline state, characterized by enhanced flexibility, while, in situ, microstructural ordering phenomena take place following stimulation, which result in a marked increase in stiffness over time. The polymer has a suitable segmental mobility at physiological conditions which allows for morphological rearrangement.

The amorphous polymer is formed by exposure to a temperature sufficiently high to melt all crystallites (e.g., 70-80° C.), followed by a very rapid quenching, for example, by immersing the material in liquid nitrogen, to solidify the material while preventing it from crystallizing

The glass transition temperature of the polymer is below 37° C. When the device is inserted into a body, it is flexible and enables smooth navigation to the site and expansion. After prolonged exposure to physiological temperatures, the polymer reverts to its crystalline state, resulting in the concomitant increase in stiffness.

In an exemplary procedure, a device is prepared comprising an amorphous polymer having the general formula:

J₁-K₁-L₁-Y-L₂-K₂-J₂

wherein:

J₁ and J₂ are each a relatively low-weight (e.g., 350 Da) polyalkylene glycol (e.g., methyl polyethylene glycol);

K₁ and K₂ are each a hydrophobic (e.g., water insoluble) segment;

L₁ and L₂ are each independently a bifunctional linking moiety or absent; and

Y is selected from the group consisting of a polyester (e.g., polycaprolactone), a polyurethane, a polyamide, a silicone polymer, a polyacrylate, a polymethacrylate, and a polyolefin, of suitable molecular weight, as described in detail hereinabove.

The polymeric component is placed in a vessel in a body, such that the device is under physiological conditions at a temperature of 37° C. When the device is in place, a balloon is inserted into the device and inflated, thereby expanding the polymeric component. Over a period of time (e.g., 20 minutes), the polymer becomes more crystalline and the device becomes stiffer, such that the expanded state of the device is maintained.

### Example 15

### In Vivo Weldable “Smart” Amorphous Cross-Linked Polymeric Components

An in vivo weldable polymeric component is prepared from an amorphous polymeric system comprising polymeric chains cross-linked by cross-linking moieties (e.g., aliphatic oligoesters) which are degradable (e.g., via enzymatic action) under physiological conditions. The polymeric chains are of a material which would be crystalline or semi-crystalline in the absence of the cross-linking moieties.

The cross-linking moieties degrade in vivo and the crystallinity in the polymeric system gradually increases over a period of time as the degree of cross-linking decreases, and the device becomes stiffer, such that the expanded state of the device is maintained.

### Example 16

In Vivo Weldable Polymeric Components with Segregating “Smart” Components

An in vivo weldable polymeric component is prepared comprising a polymeric system having at least two components. The polymeric device is placed in a vessel in a body, such that the device is. When the device is in place, a balloon is inserted into the device and inflated, thereby expanding the device.

Under physiological conditions the components then segregate over a period of time due to chemical incompatibility of the components and/or reaction products of components (e.g., products of polymerization and/or cleavage of cross-linking of the original components). For example, polymerization of a component facilitates segregation by reducing the entropy of a non-segregated mixture, and cleavage of cross-linking facilitates segregation of incompatible components by increasing molecular mobility (e.g., of a polymer chain).

As the phase blending, which inhibits crystallization, decreases, some or all of the segregated components begin to crystallize. Due to the crystallization, segregation results in a gradual increase in the stiffness of the device, such that the expanded state of the device is maintained.

### Example 17

### Additional Examples of the Preparation of Devices

### Example A

One method to prepare a balloon expandable bare metal stent for in situ welding was to dip-coat said stent in a 3% (w/w) CLUR solution in chloroform. The bare metal stents, for example, were commercially available and composed of stainless steel, were lowered into a solution manually or with a constant speed. In the case of computer-controlled dip coating, a stent was lowered into the solution with an exemplary crosshead speed of 10 mm/min In the occasion where solution remained webbed between the struts after extracting it from the solution, the solution was removed using a 21G hollow needle. Another method to remove the excess polymer was by gently blotting the relevant areas with light contact with absorbent paper. The solvent was left to vaporize under a fume hood leaving a continuous polymeric coating on the metal struts. The thickness of the coating was varied by adding additional dipping cycles, typically in the 5-15 micrometer range.

### Example B

Another method to render bare metal stents in vivo weldable, was to gently eject with a 21G hollow needle a polymer solution of 5% (w/w) CLUR in chloroform directly on the bare metal struts of the stent. As the solvent evaporated the struts were encapsulated with the polymer and there was no webbing formed between the struts spanning the open cells. Similarly, the stents were left to dry until the solvent evaporated.

### Example C

Another method to coat the struts of the bare metal stent with an in vivo weldable polymer was to exploit the Venturi effect and generate an aerosol, optionally of 2% (w/w) chloroform CLUR solution. The aerosol can be modulated to form particles on the nano or micro scale. These particles once deposited on the stent surface may coalesce to generate a homogenous layer by a moderate application of heat. This method is particularly illustrative of how one can render a non-in vivo weldable surface into an in vivo weldable surface. Surfaces that are weldable are repeatedly weldable. This includes other polymeric materials, such as PET stent-grafts, which have transition temperatures significantly higher than physiological temperatures which would make them unsuitable for in vivo welding but nevertheless were rendered in vivo weldable with the application of a thin coating.

### Example 18

### Method of Implementation

One method to implement the in vivo weldable stent grafts is to initially deliver the coated stent to the anatomically correct position with the aid of radiopaque markers located on the ends of the components of the device. After expansion of the stent, a second balloon catheter delivers an in vivo weldable polymeric component onto the stent. A suitably warm solution, typically saline, is used to warm the balloon as required and, in conjunction with the pressure from the balloon, the polymeric component is permanently bound to the stent forming a stent graft. Further stents and polymeric components may be further attached upstream or downstream of the initial stent by overlapping the sleeves. Additional configurations are also engineered using the technology disclosed hereby, as dictated by anatomical and clinical considerations.

### Additional Examples

Further illustration of the invention is presented below.

The in vivo welding concept aims at rapidly welding together two or more medical devices inside the human body, at a physiologically acceptable temperature, that will result in a strong and reliable connection.

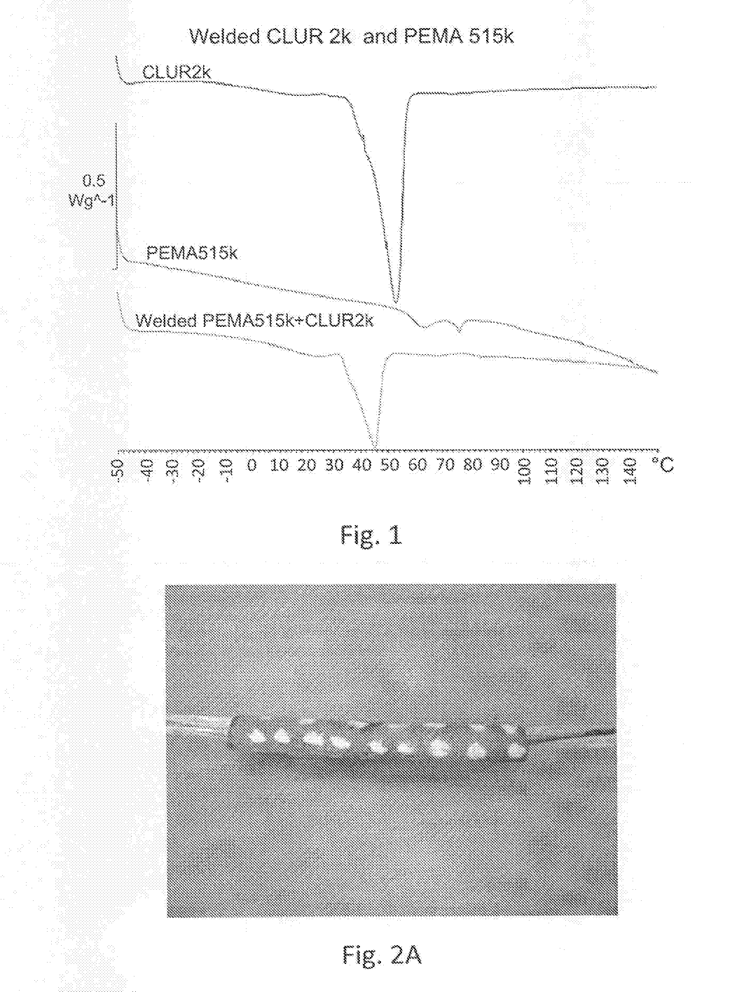

Not only the same or similar polymers were welded together, but also polymers differing substantially in ther composition and mechanical properties were successfully bonded together under physiologically acceptable conditions. The DSC thermogram shown in FIG. 1 demonstrated that the welding together of an elastomeric polyurethane (CLUR) and a stiff polymethacrylate (PEMA) succeeds to blend together the polymers at the molecular level, as shown by the disappearance and shift of the peaks in the three traces shown.

Several polymers proved to be in vivo weldable and perfomed successfully as both the in vivo weldable component/s and also the in vivo weldable polymer/s that render/s the in vivo non weldable component/s, in vivo weldable. Biostable as well as biodegradable polymers were identified. Among the former, several polyether urethanes can be mentioned. The polyethers used in this class of materials were typically polytetramethylene glycol, polypropylene glycol and polyethylene glycol of various molecular weights, among others. One example of this family, among other families, is the polyether urethane consisting of a polytetramethylene glycol (MW=650) soft segment and hexamethylene diisocyanate (HDI) as the chain extender. This polymer displays a Tᵣ at around 45-46° C., and attains an Ultimate Strength value of 42 MPa and a Young modulus of around 90 MPa.

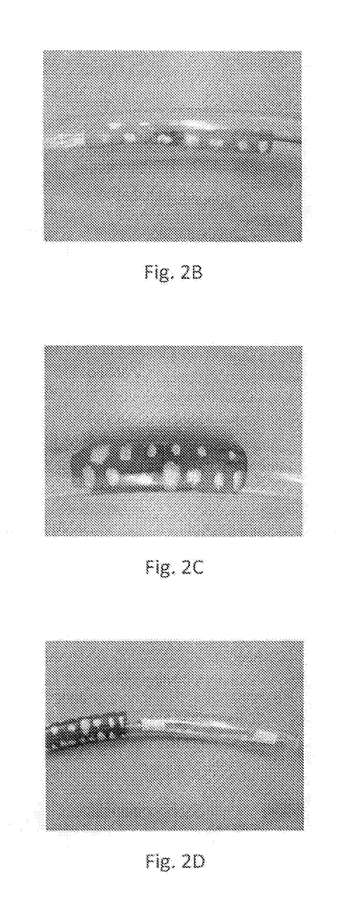

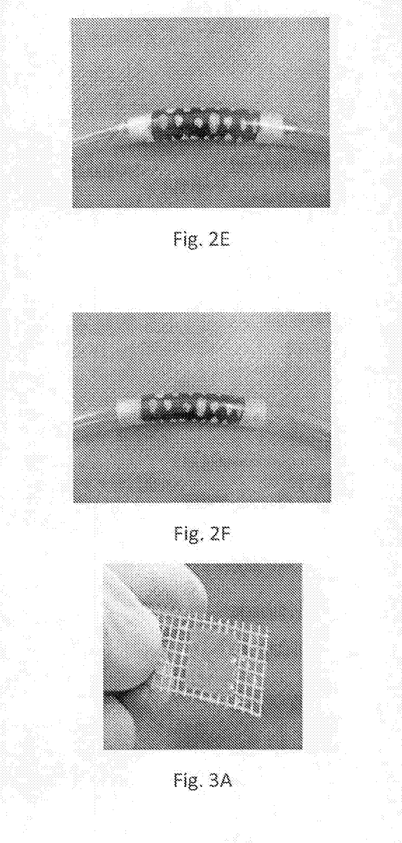

The photos of FIGS. 2A-F illustrate the in vivo welding of various in vivo weldable polymeric components of the invention, layer by layer, following the step-by-step procedure of deploying and building a thicker in vivo weldable polymeric component, from extremely thin components.

In this case, a transparent Tygon model system and a tubular thin device with white holes, is shown in FIG. 2A. In FIG. 2B, the device is inserted within the “Tygon vessel”, and in FIG. 2C this first layer of the device is easily expanded and attached to the luminal surface of the “vessel”. In FIG. 2D, the balloon is deflated and removed, while FIGS. 2E and 2F, demonstarte the deployment of a second layer, white and longer, and its subsequent welding together to the first layer already deployed and expanded at the site of performance

This procedure is conducted among various polymeric components and/or with any additional device, tissue, instrument or accesory. In some embodiments, two or more in vivo weldable polymeric components may be welded together so that they partially or totally encapsulate or “sandwich” any device, tissue, instrument or accesory.

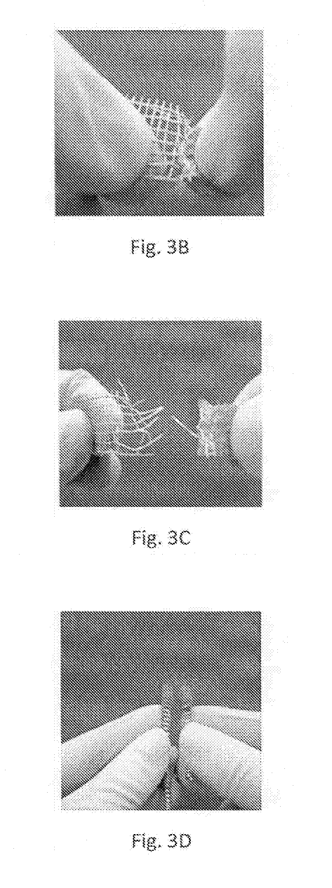

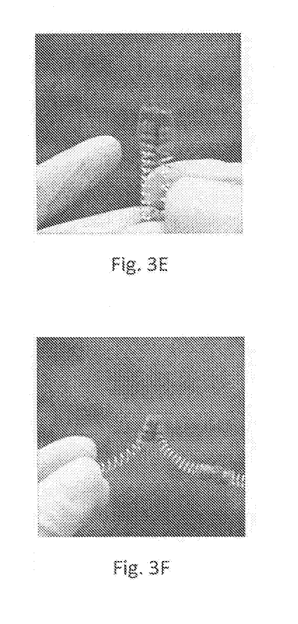

The photos shown in FIGS. 3A-C present a system constructed of two different metallic structures, one flat (FIGS. 3A-C) and one tubular (FIGS. 3D-F), conclusively proving the in vivo welding concept.

In the case of the flat metallic grid (FIGS. 3A-C), the in vivo weldable polymeric component (the square patch on the mesh) was welded to a metallic mesh, which was previously rendered in vivo weldable by coating with an in vivo weldable polymer. The coating of the mesh was achieved ex vivo, while the polymeric component was welded to the modified mesh under in vivo conditions. As apparent from the photos, the welded connection between the two was found very strong, that efforts to remove the patch, resulted in the destruction of the metallic grid.

The metallic tubular structures (FIGS. 3D-F) were chosen to mimic stents implanted throughout the vasculature and weldable connectors, in this case terminal, were added to them. Then, the “stents” were connected in series and in parallel, by rapidly and strongly welding them together via the in vivo weldable polymeric connectors. The strength of the connections was conclusively demonstrated both mechanically (see below), as well as by determining their stability under strong high volume water flow.

Since the key feature of the devices being developed is their in vivo weldability, the temperature at the device/tissue interface, is a key safety factor and was, therefore, measured.

In the experiment showed above, the temperature measured by a thermocouple at the outer surface of the device, was significantly lower than the temperature inside the balloon. In this case, while inside the balloon the water temperature was 44° C., the temperature measured at the outer surface of the device, the surface that will be in contact with the tissue, was 39° C. only. These data demonstrate that while the temperature can be sufficiently high to efficiently weld the in vivo weldable polymeric component, the tissue will be exposed to a significantly lower and physiologically acceptable temperature. It should also be stressed that the welding process, from the moment warm saline is introduced into the balloon, until the balloon is deflated and removed, takes only one-two minutes.

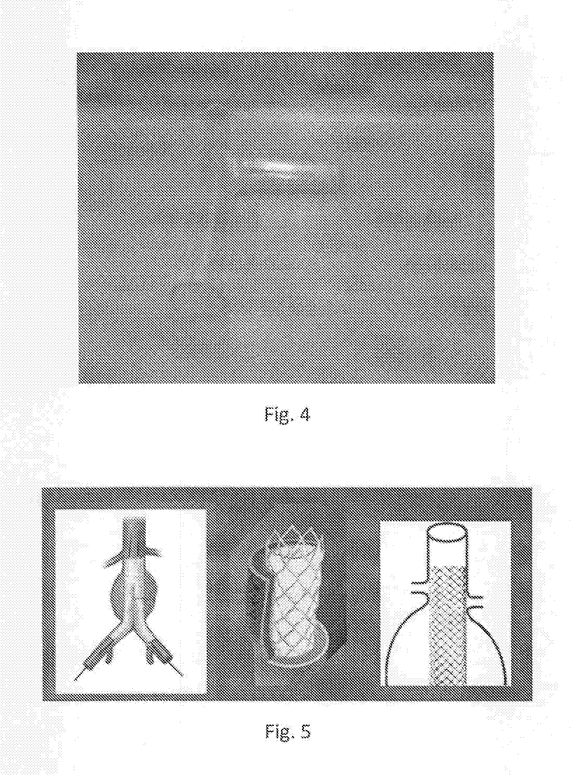

Work was also conducted using tubular polymeric model systems, with Tygon transparent tubing mimicking the vessels. FIG. 4 shows such a model system, in this case mimicking the anatomy of aorta and one renal artery.

FIG. 5, middle picture, illustrates a hybrid EVAR device comprising a metallic stent and the in vivo weldable component, deployed sequentially and welded together in vivo. The left picture illustrates the hybrid EVAR in a “pantalones” configuration, where the device is deployed in the abdominal aorta and in the iliac arteries as well. In this case, the primary role of the in vivo weldability feature is to stabilize the multicomponent structure, by also strongly welding the different devices, as well as to prevent blood leakage, due to the hermetic sealing of the joint between two stents. The right picture illustrates a scenario where infra-renal landing zones are absent.

In this very challenging case, the metallic stent is deployed first, protruding supra-renally, and then the in vivo weldable device is deployed, so that it strongly welds to the metallic stent, without blocking the blood flow from the aorta into the renal arteries. Furthermore, the in vivo weldable component can be tailored at the OR, as dictated by the specifics of the anatomy of the patient. These are three of the most challenging scenarios, where the advantageous features of the devices disclosed hereby, play a cardinal role. The in vivo weldable devices of this invention, not only improve the outcome of the procedure but also significantly expand the scope of application of the EVAR technology, making patients that would have had to undergo open surgery or did not have any other modality of treatment available to them, eligible to undergo the minimally invasive EVAR procedure. In other embodiments, more than one device may be deployed sequentially, in any order, as the peculiarities of each case demand In some aspects of other embodiments where two or more devices have to be deployed in a head-to-tail configuration, each of the devices may be deployed sequentially, optionally the in vivo weldable metallic stent having its struts coated with an in vivo weldable polymer being deployed first and firmly positioned at its site of performance, followed by the deployment of the in vivo weldable polymeric sleeve, and the in vivo welding of both components is conducted. In some embodiments, the in vivo weldable polymeric sleeve may be somewhat longer than the stent, so it protrudes longitudinally, allowing to weld the two or more devices positioned in series, to be welded together. In yet another embodiment, the in vivo weldable metallic stent having its struts coated with an in vivo weldable polymer is deployed initially and securely placed at its site of performance, followed by the deployment of the in vivo weldable polymeric sleeve, and then both components are in vivo welded, with the distal end of one stent touching or very close to the proximal end of the other stent. In this embodiment an in vivo weldable polymeric connector is deployed bridging over the gap between the two stents, and strongly connecting them together. Said gap between the two stents can be inexistent, in which case the stents touch each other, up to being of macroscopic dimensions, depending on the site and the particular requirements dictated by each clinical case. In yet other embodiments, two or more of the devices may be deployed when in a side-by-side configuration, as in the common “kissing stents” case. In some aspects of these embodiments, an external, longitudinal connector will be deployed between the two “kissing stents”, securely in vivo welding the two.

In yet other aspects of these embodiments, two or more of the devices may be deployed when in a branched configuration, and in vivo welded together via in vivo weldable protrusions of the in vivo weldable polymeric component in vivo welded to each of the two or more stents, said protrusions allowing the strong and firm connection between the different devices. In yet other embodiments, in vivo weldable polymeric connectors are deployed at the junction between two stents, and strongly connecting them together at the in vivo weldable junction between them. In some embodiments, other procedures based on in vivo welding

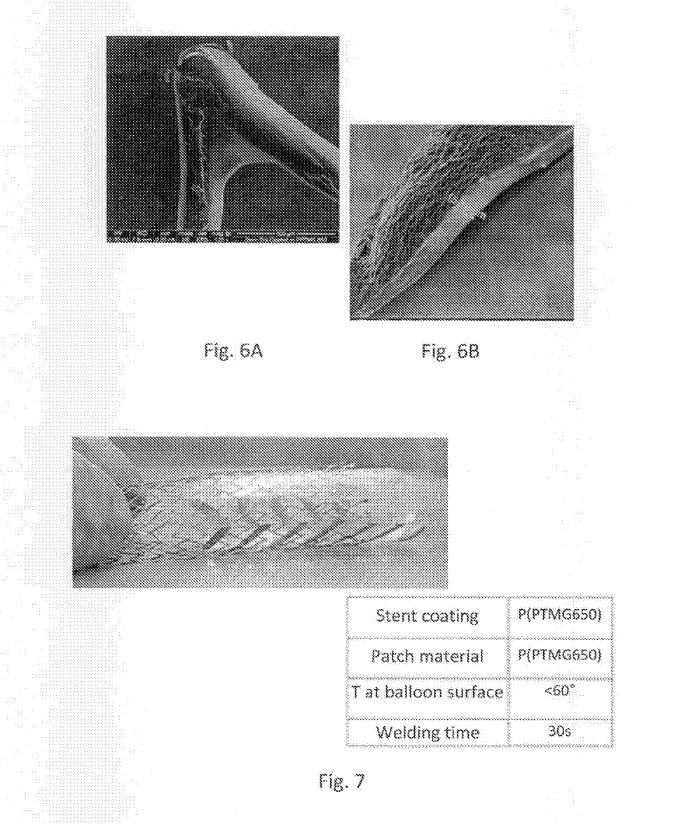

In FIG. 6A, a coating of a strut of a stent with an in vivo weldable polymer is shown under amplification, and in FIG. 6B the thickness of the coating (in this case, around 15 micrometers) is shown.

FIG. 7 shows the welding of an in vivo weldable patch (with stripes, for visualization purposes) and a metallic stent, with its struts coated with the same polymer (see table in FIG. 7). The patch illustrates not only the deployment of a patch, but also that of any other in vivo weldable polymeric component.

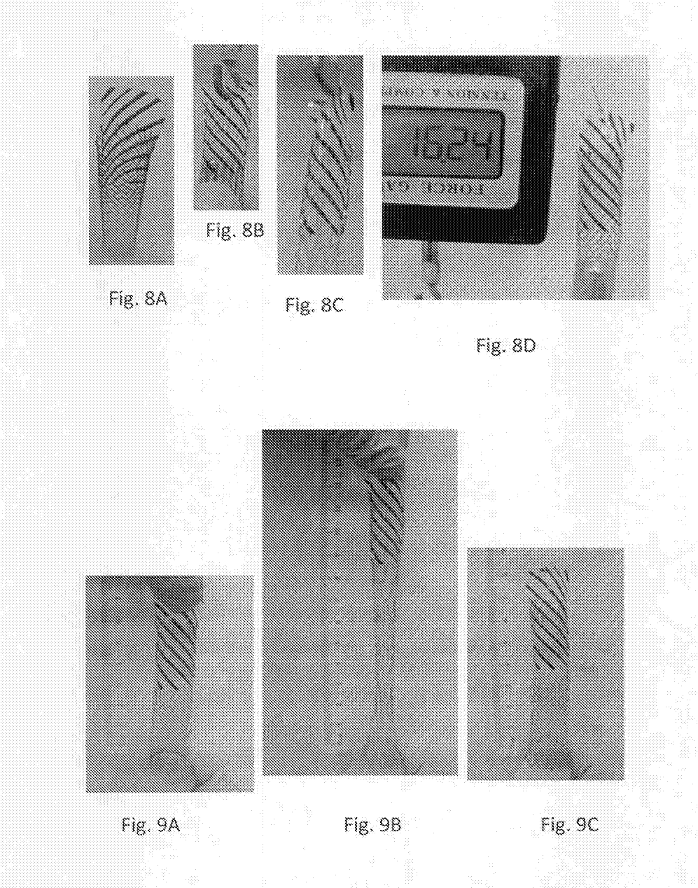

FIGS. 8A-D follow the measurement of the force required to pull out the patch, welded to the stent (FIGS. 8A-B), using a tensiometer (FIG. 8C). As apparent from FIG. 8D, the patch finally failed at a load of around 16 N, ruptured at the hole done to introduce the hook of the tensiometer. To any versed in the art it is evident that a huge stress concentration takes place at the hole done to introduce the hook of the tensiometer. It is also worth noticing that the welding bond did not fail.

FIGS. 9A-C show the manual extension of the patch welded to the stent, up to around 300% elongation, with the welding connection staying in place, under those harsh conditions.

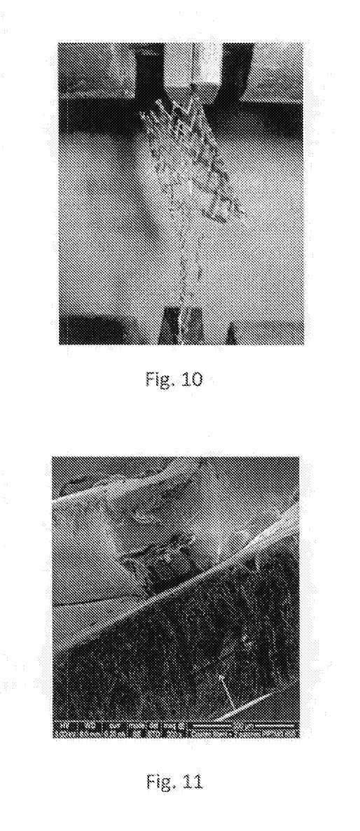

FIG. 10 shows the last stage of a mechanical testing of the strength of the welding bond between the stent and the patch. As is apparent from the photo, it was the metallic stent itself that failed, with the welding bond between the patch and the stent sustaining the large stresses applied.

The SEM micrograph shown in FIG. 11, presents the coated strut and its welding to an in vivo polymeric patch and said patch is welded to a second one. The efficiency of the welding process is apparently shown.

Another system according to the invention is constructed of a Tygon “vessel” and three in vivo weldable components, that were welded endoluminally, within the Tygon “vessel”. In a kit of the invention, certain components were deployed endoluminally, being separated by a distance of around 3 mm Then, an in vivo weldable component was deployed and welded together via an in vivo weldable connector deployed across a 3 mm gap.

Enabling a surgeon to follow closely and accurately the two components of the device, e.g., a stent and a sleeve, throughout the whole deployment procedure, is of the utmost importance. In light of the above, it was imperative rendering the sleeve radiopaque, and doing so in a manner that does not hamper any of its performance requirements. In various embodiments of this invention, therefore, radiopaque sleeves were produced by adding a radiopaque agent such as, without limitation, BaSO₄ or ZrO₄, among several others. Among them, when the sleeve is prepared following the dip coating technique or a similar one, to add particles of the radiopaque agent to the dip coating solution. The same applies if the sleeve is prepared by extrusion and similar processing techniques. In all these cases the radiopaque particles, may be added prior to or during the production or immediately after, when the sleeve is still soft and somewhat tacky, due to the presence of some solvent, in techniques involving solvents, or heat, in those methods where heat is used during the manufacture of the sleeve. Another method implemented and disclosed hereby for the generation of radiopaque sleeves focuses on “stamping” the radiopaque agent, such as BaSO₄, among others, to produce radiopaque sites, such as dots and bands, at different positions along the sleeve such as, without limitation, its proximal and distal ends. This can be achieved by slightly heating the sleeve and applying pressure to the radiopaque powder and the somewhat softened polymer. Optionally, the softening can also be achieved by using a proper liquid, able to suitably soften the polymeric sleeve, enabling the efficient “stamping” of the radiopaque agent. Additional approaches taught by the present invention included using radiopaque markers and attaching them to the sleeve by, for example, without limitation, welding them to the polymeric sleeve by means of a small weldable patch. This forms a “sandwich” configuration, with the marker, typically tantalum, being in the middle, between the sleeve and the patch, as shown below.

Another method disclosed hereby was to incorporate a radiopaque fiber or wire at selected locations in the sleeve, by any technique such as, without limitation, passing through the radiopaque string/lace/ribbon/yarn/ through the sleeve.

Since in many instances the sleeve will directly interface with blood, and, therefore, typically, being satisfactorily non-thrombogenic is a crucial requirement and, therefore, any method able to achieving this goal, is applicable to the sleeves disclosed hereby. These methods include entrapping by chemical or physical or biological means molecules able to improve the blood compatibility of the sleeves, said molecules being released from the sleeve over time. Heparin is one example of the plethora of molecules able to perform this task. These bioactive molecules may be directly dispersed within the sleeve, or in any other configuration such as, without limitation, as aggregates, or encapsulated in nano or microparticles of any geometry, or any other format that will allow the optimal release of said molecules, and combinations thereof. Additionally, said molecules can be physically and/or chemically and/or biochemically attached to the surface of the sleeve. In the alter embodiments, said species can bepermanetly attached to the surface and/or they can be released over time. Given their well-recognized ability to minimize protein adsorption and cell attachment on surfaces, polyethylene glycol (PEG) chains is one of the molecules covalently grafted to the sleeve surface. One of the surface grafting schemes performed, consisted of exposing the sleeve surface to plasma of ammonia, whereby amine moieties were generated. These amine groups performed as reactive anchoring sites and were reacted with difunctional molecules, such as, without limitation diisocyanates, such as hexamethylene diisocyanate (HDI), which, in turn, reacted with the PEG chains, via their terminal OH groups. In some instanced themolecules is more than bifunctional.

The occurrence of the plasma treatment was determined by contact angle measurements. The initial contact angle of CLUR polymers, around 80°, substantially decreased to around 40°, due to the presence of the hydrophilic amine groups on CLUR′ surface, after its exposure to plasma of ammonia.

Additionally, PEG molecules of different molecular weights were end-capped with one terminal C═C double bonds by reacting them, with isocyanatoethyl methacrylate (IEMA), for example, whereby the corresponding methacrylate was formed, as shown below. This surface modification scheme was performed using PEG chains of various molecular weights and generating different surface densities.

The success of the addition of double bonds to PEG1000, 2000 and 3400 was evaluated by H-NMR analysis, which demonstrated that in all cases, there was one double bond per PEG chain.

Once the PEG methacrylates were synthesized, the double bond was reacted with the NH₂ moieties generated by the plasma of ammonia on the surface of the sleeve, through the Michael addition reaction.

After carrying out the Michael addition, the samples were thoroughly washed and then studied by performing contact angle measurements and XPS analysis.

Percutaneous Implantation of Covered Stent Devices into Abdominal Aorta and Iliac Arteries in Pigs

A medium size (typically between ˜40-60 Kg) pig model was chosen for investigating the performance of the devices disclosed hereby, based on the performance of a series of CT angiographic studies in animals weighing

Typically the surgical procedure was as follows. Using Ultrasound guided bilateral femoral artery access with commercially available vascular access devices, catheter angiography of the abdominal aorta and pelvic arteries was performed. In one animal, three commercially available “Advanta V12” (Atrium Medical Corporation) covered stents were inserted, one in the distal abdominal aorta, and one in each of the common iliac arteries. All three devices were inserted in a simple linear configuration. In a second specimen, four Advanta 12 covered stents were inserted, two overlapping in the distal abdominal aorta, and one in each of the common iliac arteries with proximal overlap with the lower stent in the aorta together with “kissing” of the two iliac stents.

Both animals were then maintained in the animal facility according to ethically acceptable practice for a period of approximately six weeks. At that point in time, the terminal experiment was performed, again “back to back”, on one day. After the induction of general anesthesia, the animal was transported to the CT scanner, where CT angiography was performed to evaluate stent positioning, evidence of vascular “injury” or presence of “neo-intimal hyperplasia”. The configuration observed demonstrated overlapping stents in the aorta and smaller caliber stents overlapping and “kissing” within the larger aortic device.

The axial images of the CT angiography were evaluated for reasons described above. Multi-planar and volumetric reconstruction of the CT angiography images was subsequently performed for the purpose of data evaluation and presentation.

The sleeping animal was then returned to the animal lab where the abdominal aorta and proximal pelvic arteries were surgically explanted in a terminal procedure. The specimens were photographed and examined for macroscopic evidence of major vascular injury (inflammation, dilatation, perforation, adhesion, etc.). The specimens were labeled and placed in 10% formalin for subsequent pathological evaluation.

### Biocompatibilty and Thrombogenicity Study of the Sleeve

The biocompatibility study will be conducted at HARLAN Laboratories Inc., in Rechovot and the thrombogenicity analysis was performed in collaboration with the Department of Hematology, Coagulation Lab at the Hadassah Medical Center. The polymer component study material was evaluated using standard mechanisms that assess thrombogenicity in absolute and relative terms (i.e. in vitro comparison to other materials with known degrees of thrombogenicity).

The animal experiments were conducted on ˜50 kilogram female pigs, under general anesthesia, using bilateral femoral artery access. 8 Fr. vascular sheaths were used and the devices were implanted along the aorta and iliac arteries of the animal The animal received aspirin 100mg/day post-operatively.

A one week post insertion angiogram showed stable position of both stent and sleeve, suggesting durable and stable welding. Also, no significant stenosis was observed and thromogenicity profile was good. Furthermore, normal flow was observed and all branches were open. Follow up angiography performed after 38 days demonstrated normal flow and no stenosis of note. The animal was healthy with no signs of ischemia.

1-31. (canceled)

32. A kit for in vivo assembly of an implantable article, the kit comprising two or more article segments suitable for assembly into said article, in vivo, at least one of said article segments comprising a material selected from the group consisting of a metal, a ceramic material and a polymer having a softening temperature above 40° C., at least one segment having at least one material region selected of a weldable material, the kit further comprising instructions for assembling said article in vivo.

33. The kit according to claim 32, wherein each of said article segments has at least one material region selected of a weldable material.

34. The kit according to claim 32, wherein each of said article segments comprises a material region of a material selected from the group consisting of a metal, a ceramic material and a polymer having a softening temperature above 40°, and has at least one material region selected of a weldable material.

35. The kit according to claim 32, wherein at least one of the segments is constructed entirely of a weldable polymeric material.

36. The kit according to claim 32, wherein each of said article segments is suited for in vivo welding via each of said at least one material region selected of a weldable material present on each segment.

37. The kit according to claim 32, wherein each article segment comprises at least one region of at least one weldable material.

38. The kit according to claim 32, wherein each article segment is appended or associated with at least one weldable material, said weldable material having been associated with said article segment ex vivo.

39. The kit according to claim 32, wherein one or more of the article segments is manipulated ex vivo to receive thereonto at least one polymeric material, and each of the remaining article segments comprises at least one region of at least one weldable material.

40. The kit according to claim 32, wherein one or more of the article segments is manipulated ex vivo to receive thereonto at least one polymeric material, and each of the remaining article segments is of at least one weldable material.

41. The kit according to claim 32, wherein the weldable material is selected from the group consisting of polymeric materials having a softening temperature between about 37 and 40° C.

42. An implantable article constructed or comprising a material selected from the group consisting of a metal, a ceramic material and a polymer having a softening temperature above 40° C., said article being modified to receive, by welding, onto at least a region thereof an element comprising a polymeric material, wherein welding is achieved in vivo.

43. The article according to claim 42, wherein said at least a region is associated with a polymeric material, said polymeric material having been associated with said region ex vivo, said element being capable of welding to said element in vivo.

44. The article according to claim 43, wherein said polymeric material is associated with a region on said implantable article which is resistant to in vivo welding.

45. An implant when positioned in an animal body, the implant comprising a material region constructed or comprising a material selected from the group consisting of a metal, a ceramic material and a polymer having a softening temperature above 40° C., said material region being associated with a first material, said first material being associated with a second material, wherein association of said region with the first material is achieved ex vivo and wherein association of said first material with said second material is achieved in vivo.

46. The article according to claim 45, being in a form selected from the group consisting of an implant, a device, a system, a prosthesis, an instrument and an accessory.

47. The article according to claim 46, having a metallic backbone.

48. The article according claim 47, associated with at least one weldable polymeric material, said material being fused, welded, incorporated, attached, woven, knitted, braided, or coated, ex vivo, to a surface region of the article.

49. The article according to claim 48, wherein the polymeric material is a polymer formed of an oligomer selected from the group consisting of ethylene glycol dimethacrylate EGDMA, triethylene glycol dimethacrylate TEGDMA, polyethylene glycol PEG 600diacrylate, polypropylene glycol PPG500diacrylate, PEG 600dimethacrylate, PPG500dimethacrylate, double-bond end capped PEG/PPG diblocks and triblocks, low molecular weight polyamides, polyurethanes and polyesters.

50. The article according to claim 42, wherein the weldable material is selected from the group consisting of polymeric materials having a softening temperature between about 37 and 40° C.

51. A method for assembling an implantable article in vivo, the method comprising:
delivering into a body region a first segment of said article and positioning said segment at a desired position;
delivering, in sequence, one or more segments; and
in vivo welding each segment to a previous segment by welding;
wherein at least of said article segments is constructed of or comprises a material selected from the group consisting of a metal, a ceramic material and a polymer having a softening temperature above 40° C., and having at least one material region selected of a weldable material,
wherein welding is achieved by softening said at least one material region on each of two of the segments to be welded, permitting association of each of the article segments and article assembly; and
wherein, optionally, the first segment of said article is constructed of a material selected from the group consisting of a metal, a ceramic material and a polymer having a softening temperature above 40° C., and having at least one material region selected of a weldable material.
